(19) United States
(12) Patent Application Publication
(10) Pub. No.: US 2014/0294815 A1
(43) Pub. Date: Oct. 2, 2014

(54) CANINISED TUMOUR NECROSIS FACTOR ANTIBODIES AND METHODS OF USING THE SAME

(71) Applicant: David Gearing, Southbank (AU)

(72) Inventor: David Gearing, Southbank (AU)

(73) Assignee: NVIP Pty Ltd, Melbourne, Victoria (AU)

(21) Appl. No.: 14/241,616

(22) PCT Filed: Aug. 29, 2012
(86) PCT No.: PCT/GB2012/052115

§ 371 Date: Jun. 4, 2014

Related U.S. Application Data
(60) Provisional application No. 61/529,020, filed on Aug. 30, 2011.
(60) Provisional application No. 61/531,439, filed on Sep. 6, 2011.

(51) Int. Cl. C07K 16/24

(52) U.S. Cl. CPC C07K 16/241
USPC 424/133.1; 530/387.3; 435/69.6

(57) ABSTRACT

Caninised and chimeric antibodies and antigen binding fragments thereof which bind specifically to canine tumour necrosis factor and inhibit the ability of canine TNF to bind to the TNFR1 receptor are provided. The invention further extends to nucleic acids encoding same and to methods of treating chronic inflammatory disease such as arthritis in a canine using said antibodies and/or nucleic acids.

### FIELD OF THE INVENTION

The present invention relates to binding antibodies and fragments thereof which act as inhibitors of canine tumour necrosis factor alpha. The invention extends to methods of preparing same and to the therapeutic use of these binding antibodies and fragments in treating chronic inflammatory conditions mediated in canines by tumour necrosis factor, such as arthritis.

### BACKGROUND TO THE INVENTION

Tumour necrosis factor alpha (TNF-alpha, TNF-α, TNF) is a potent cytokine that exerts pleiotropic functions in immunity, inflammation, control of cell proliferation, differentiation and apoptosis. The inhibition of TNF using tumour necrosis factor receptor (TNFR)-immunoglobulin Fc domain fusion proteins or anti-TNF neutralising monoclonal antibodies has been proven to be a successful therapeutic approach to treat a variety of human inflammatory diseases including rheumatoid arthritis and psoriatic arthritis.

Companion animals such as dogs develop similar inflammatory diseases, including osteoarthritis, immune-mediated polyarthritidies, plasmatic-lymphocytic synovitis, systemic lupus erythematosis (SLE), vasculitis and a variety of autoimmune skin diseases. It is estimated that one in five adult dogs in the USA has arthritis and dogs have been used as models of human joint disease, e.g. for osteoarthritis, anterior cruciate ligament disruption and meniscal damage.

The role of TNF in the occurrence of inflammation in dogs has been extensively documented. For example, the treatment of dogs with the TNF inhibitor etanercept (huTNFR-Fc) reduced myocardial injury by approximately 25-40% following ischaemia-reperfusion induced by balloon occlusion in a closed chest model, with concomitant reduction in associated inflammatory markers such as ICAM-1 and NF-kB. Similarly, a 60% reduction in infarct size in an open chest dog model of ischemia reperfusion using 2 mg/kg TNFR-Fc has also been demonstrated.

Further, there is increased secretion of TNF alpha in cell infiltrates of synovial fluid of dogs presenting with stifle arthritis. TNF and the type II TNF receptor have been shown to be significantly elevated in central and peripheral retina of dogs with glaucoma as part of a broad inflammatory response. Anti-canine TNF MAbs have been used to detect low levels of TNF by capture ELISA in supernatants of canine PBMC treated with LPS. TNF alpha expression has also been reported in skin samples of canine hemangiopericytoma, tricoblastoma, lipoma and mastocytoma.

TNF-alpha and TNF receptors are present in canine articular cartilage in an induced model of osteoarthritis. Adalimumab, the humanised monoclonal antibody to TNF, has been tested in two dogs with exfoliative cutaneous lupus erythematosus (ECLE) (0.5 mg/kg every 2 weeks for 8 weeks), but the disease progress was unaltered and serum TNF-alpha levels were unchanged.

Accordingly, due to the involvement of TNF in a wide range of inflammatory mediated conditions in the canine there is a need for inhibitors of canine TNF that can be used in the long-term inhibition of TNF in order to prevent or treat said chronic inflammatory conditions.

### SUMMARY OF THE INVENTION

Following extensive efforts, the present inventor has surprisingly identified a method for preparing antibodies which produces antibodies and binding fragments which bind specifically to canine TNF and which neutralise the biological activity of canine TNF. In particular, it is demonstrated herein that despite introducing a number of amino acid modifications into the framework region of the antibody, because of the novel and inventive way these modifications are made, the antibodies and binding fragments of the invention bind to canine TNF with high specificity. It is further demonstrated that binding sequesters the biological activity of canine TNF by inhibiting the binding of canine TNF to cell membrane expressed TNF receptors. This, in turn, will enable prevention of the occurrence of a TNF mediated induction, development or progression of inflammatory mediated diseases in canines, such as arthritis.

The antibodies of the invention are produced using recombinant DNA methods and exhibit binding specificity for canine TNF, whilst also having canine framework and constant domain sequences to reduce their immunogenicity when administered to a canine host. As a result, the risk of xenoantibody induction is minimised. The binding specificity for canine TNF is entirely surprising and unexpected as the antibodies were not produced using standard methodologies, such as CDR grafting or the like.

According to a first aspect of the invention there is provided a caninised antibody or antigen binding fragment thereof which binds specifically to canine tumour necrosis factor (TNF).

In certain embodiments, the antibody is prepared by a method comprising or consisting essentially of the steps of:
providing a donor antibody from a species other than a canine, wherein the donor antibody has binding specificity for tumour necrosis factor;
comparing each amino acid residue of the amino acid sequence of framework regions of the donor antibody with each amino acid residue present at a corresponding position in the amino acid sequence of framework regions of one or more canine antibodies to identify one or more amino acid residues within the amino acid sequence of the framework regions of the donor antibody that differ from one or more amino acid residues at the corresponding position within the amino acid sequence of framework regions of the one or more canine antibodies; and
substituting the one or more identified amino acid residues in the donor antibody with the one or more amino acid residues present at the corresponding position in the one or more canine antibodies.

The above method modifies a donor antibody for use in a canine in such a way that the modified antibody does not contain any amino acid in any position within the framework regions which would be foreign at that position in canines. The modified antibody therefore retains the specificity and affinity of the donor antibody for the target antigen, but importantly is modified such that no potentially foreign epitopes are created. The modified antibody is therefore not seen as foreign in canines and hence does not induce an immune response in canines which could lead to a neutralisation of the efficacy of the antibody, especially following long term administration.

In certain embodiments, the step of substituting the one or more identified amino acid residues comprises substituting the one or more identified amino acid residues with the one or more amino acid residues present at the corresponding position which have the highest homology to the one or more substituted amino acid residues.

In certain embodiments, the method further comprises the step of replacing constant domains of the heavy chain and/or light chain of the donor antibody with constant domains of a heavy and/or light chain derived from a canine antibody.

The above method does not comprise CDR grafting. Antibodies prepared according to the above method comprise CDRs of the donor antibody, caninised framework regions prepared according to the above method and canine constant domains.

The present invention extends to antibodies prepared according to the above method such as those described below and fragments thereof.

Accordingly, according to a further aspect of the invention there is provided a composition comprising a peptide, or a nucleic acid sequence encoding a peptide, wherein the peptide comprises an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9. Typically, the peptide binds tumour necrosis factor alpha. In certain embodiments, the peptide is a caninised or chimeric antibody or a binding fragment thereof. In certain embodiments, the caninised antibody is prepared according to the method of preparing an antibody described above.

In certain aspects, the present invention provides compositions comprising a canine tumour necrosis factor alpha binding agent, or an oligonucleotide encoding a tumour necrosis factor alpha binding agent, where said binding agent comprises an amino acid sequence selected from the group consisting of SEQ ID NO:1, SEQ ID NO:2, SEQ ID NO:3, SEQ ID NO:4, SEQ ID NO:5, SEQ ID NO:6 SEQ ID NO:7, SEQ ID NO:8 and SEQ ID NO:9.

A yet further aspect of the invention provides a caninised or chimeric antibody or an antigen binding fragment thereof which binds specifically to canine tumour necrosis factor (TNF), in particular canine TNF alpha (TNF-α). Typically, the caninised or chimeric antibody, or the antigen binding fragment derived therefrom, neutralises the biological activity of canine TNF. In particular, the binding of the caninised or chimeric antibody or binding fragment to canine TNF sequesters the ability of canine TNF to bind and activate membrane-associated canine TNF receptors. In certain embodiments, the caninised or chimeric antibody comprises an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9. In certain embodiments, the caninised antibody is prepared according to the method of preparing an antibody described above.

A yet further aspect of the invention provides a caninised antibody which binds to complement and initiates complement-mediated lysis of TNF-alpha expressing cells. Such antibodies will reduce the number of TNF expressing inflammatory cells and so result in long term anti-inflammatory activity in vivo. In certain embodiments, the caninised antibody is prepared according to the method of preparing an antibody described above.

In certain embodiments of the above aspects of the invention, the caninised or chimeric antibody, or binding fragment thereof, binds to TNF with a binding affinity of K_{D} of 1×10⁻⁸ or less.

In a further or related aspect of the invention, there is provided a neutralising antibody, or an antigen binding fragment thereof, which is capable of specifically binding to canine tumour necrosis factor alpha (TNF-alpha), the antibody or antibody binding fragment comprising, consisting of or consisting essentially of a light chain variable domain comprising the amino acid sequence of SEQ ID NO:1, or an amino acid sequence which has an identity of at least 85, 90, 95 or 99% thereto, and/or a heavy chain variable region comprising the amino acid sequence of SEQ ID NO:2, or an amino acid sequence which has an identity of at least 85, 90, 95 or 99% thereto. Typically, the antibody is a chimeric or caninised antibody.

In certain embodiments where the antibody is a caninised antibody or a fragment thereof, it comprises, consists of or consists essentially of a light chain comprising the amino acid sequence of SEQ ID NO:3 or an amino acid sequence which has an identity of at least 85, 90, 95 or 99% thereto.

In certain embodiments, the variable region of the heavy chain (VH) is conjoined to a further amino acid sequence which comprises at least one immunoglobulin constant domain. In certain embodiments, the immunoglobulin constant domain is derived from an antibody of the subclass IgG (immunoglobulin G) to form the complete heavy chain of the caninised antibody of the invention. Four different canine heavy chain constant domains are known. Typically, said constant domains comprise CH1, CH2 and CH3 regions along with a suitable linker (or “hinge”) located between the CH1 and CH2 regions.

In certain embodiments, the caninised antibody or antibody binding fragment comprises, consists of or consists essentially of a heavy chain comprising the amino acid sequence selected from the group consisting of SEQ ID NO:4, SEQ ID NO:5, SEQ ID NO:6 and SEQ ID NO:7, or an amino acid sequence which has a sequence identity of at least 85, 90, 95 or 99% thereto. In certain embodiments, the chimeric antibody or antibody binding fragment comprises, consists of or consists essentially of a heavy chain comprising the amino acid of SEQ ID NO:9 or an amino acid sequence which has a sequence identity of at least 85, 90, 95 or 99% thereto. In certain embodiments, the chimeric antibody comprises a heavy chain having the amino acid sequence selected from the group consisting of SEQ ID NO:20, SEQ ID NO:21 and SEQ ID NO:22, or an amino acid sequence having at least 85%, 90% or 95% sequence homology thereto.

In embodiments where the antibody is a caninised antibody, the antibody has an amino acid sequence wherein the residues have been altered by deletion, addition and/or substitution to de-immunise the sequence such that xenoantibodies will not be produced there against when administered to a canine subject. In particular, the antibodies comprise canine derived framework and constant domain amino acid sequences.

In a further aspect, the present invention extends to a neutralising antibody, or an antigen binding fragment thereof, which specifically binds to canine tumour necrosis factor (TNF), the antibody or antibody binding fragment comprising, consisting of or consisting essentially of a light chain and a heavy chain wherein the variable region of the light chain (VL) comprises an amino acid sequence of SEQ ID NO:1 or an amino acid sequence which has a sequence identity of at least 85, 90, 95 or 99% thereto, and wherein the variable region of the heavy chain (VH) comprises, consists or consists essentially of an amino acid sequence which is identical or substantially homologous to the amino acid sequence of SEQ ID NO:2 or an amino acid sequence which has a sequence identity of at least 85, 90, 95 or 98% thereto.

In certain embodiments, the antibody is a caninised antibody which comprises a light chain which comprises, consists of or consists essentially of the amino acid sequence of SEQ ID NO:3 and/or a heavy chain which comprises, consists of or consists essentially of an amino acid sequence selected from the group consisting of SEQ ID NO:4, SEQ ID NO:5, SEQ ID NO:6 and SEQ ID NO:7, or a sequence having an amino acid identity of at least 85%, more preferably of 95% and most preferably at least 98% identity thereto. Typically the light chain is a kappa light chain.

In certain embodiments, the antibody is a chimeric antibody which comprises a light chain which comprises, consists of or consists essentially of the amino acid sequence of SEQ ID NO:8, or a sequence having an amino acid identity of at least 85%, more preferably of 95% and most preferably at least 98% identity thereto, and/or a heavy chain which comprises, consists of or consists essentially of an amino acid sequence of SEQ ID NO:9, or a sequence having an amino acid identity of at least 85%, more preferably of 95% and most preferably at least 98% identity thereto. Typically the light chain is a kappa light chain.

In further embodiments, the antibodies or binding fragments of the invention extend to aglycosylated variants, wherein the constant domain amino acid residues which are suitable for glycosylation are deleted or substituted with a residue which cannot be glycosylated, in order to prevent glycosylation.

The inventor has further defined a series of framework regions (FR) which can be combined with complementarity determining regions (CDRs) to form caninised heavy and light chain variable domains. Each of the heavy and light chain domains has 4 framework regions, designated FR1, FR2, FR3 and FR4.

An antibody molecule may comprise a heavy chain variable domain comprising CDR1, CDR2 and CDR3 regions along with associated interposed framework regions. The heavy chain variable domain (VH) CDRs are known as VHCDRs, with these CDRs being found at the following positions according to the Kabat numbering system: VHCDR1—Kabat residues 31-35, VHCDR2—Kabat residues 50-65, VHCDR3—Kabat residues 95-102 (Kabat E A et al. (1991) Sequences of proteins of immunological interest, 5th edition. Bethesda: US Department of Health and Human Services). Furthermore, an antibody further comprises a light chain variable domain comprising CDR1, CDR2 and CDR3 regions and associated interposed framework regions. The light chain variable domain (VL) CDRs are known as VLCDRs, with these CDRs being found at the following amino acid residue positions according to the Kabat numbering system: VLCDR1—Kabat residues 24-34, VLCDR2—Kabat residues 50-56, VLCDR3—Kabat residues 89-97. A light or heavy chain variable domain comprises four framework regions, FR1, FR2, FR3 and FR4, interposed with CDRs in the following arrangement: FR1-CDR1-FR2-CDR2-FR3-CDR3-FR4.

In a yet further aspect, the present invention extends to an anti-canine TNF antibody, or a canine TNF binding fragment thereof, the antibody or antibody binding fragment comprising a light chain variable region comprising at least one of:
a FR1 framework region consisting of or comprising the amino acid sequence of SEQ ID NO:10,
a FR2 framework region consisting of or comprising the amino acid sequence of SEQ ID NO:11,
a FR3 framework region consisting of or comprising the amino acid sequence of SEQ ID NO:12, and
a FR4 framework region consisting of or comprising the amino acid sequence of SEQ ID NO:13,
   and/or a heavy chain variable region comprising at least one of:
a FR1 framework region consisting of or comprising the amino acid sequence of SEQ ID NO:14,
a FR2 framework region consisting of or comprising the amino acid sequence of SEQ ID NO:15,
a FR3 framework region consisting of or comprising the amino acid sequence of SEQ ID NO:16, and
a FR4 framework region consisting of or comprising the amino acid sequence of SEQ ID NO:17.

Typically the light and heavy chain CDRs are derived from an antibody which has binding specificity to canine TNF.

Typically, the production of the caninised anti-canine TNF antibody of the invention results from a recombinant DNA process which does not require back mutations to be introduced into the framework regions of the light or heavy chain variable domains.

In certain embodiments, the light chain variable domain comprising said at least one framework region described above is conjoined to a canine derived light chain constant domain, typically a light chain kappa constant domain, but optionally a lambda light chain. In certain embodiments, said light chain comprises an FR1 region with the amino acid sequence of SEQ ID NO:10, an FR2 region with the amino acid sequence of SEQ ID NO:11, an FR3 region with the amino acid sequence of SEQ ID NO:12, and an FR4 region with the amino acid sequence of SEQ ID NO:13 or a framework region with an amino acid sequence which has a sequence identity of at least 60, 70, 80, 90, 95 or 98% to the foregoing. In certain embodiments said identity is over a length of at least about 5 amino acids, preferably about 10 amino acids.

In certain further embodiments, the heavy chain variable region comprising at least one of the framework regions described above is conjoined to a canine derived heavy chain constant domain. In certain embodiments, the amino acid sequence of the constant domain lacks any post-translational modifications, or may be modified to remove any or all residues which may be subject to N-linked or O-linked glycosylation, such that the constant domains are aglycosylated. In certain embodiments the heavy chain comprises an FR1 region with the amino acid sequence of SEQ ID NO:14, an FR2 region with an amino acid sequence of SEQ ID NO:15, an FR3 region with an amino acid sequence of SEQ ID NO:16 and an FR4 region with an amino acid sequence of SEQ ID NO:17 or a framework region with an amino acid sequence which has a sequence identity of at least 60, 70, 80, 90, 95 or 98% to the foregoing. In certain embodiments said identity is over a length of at least about 5 amino acids, preferably about 10 amino acids.

In certain further embodiments, modifications may be made to the framework regions described herein. That is, the inventor has identified that for some residues in each framework region, there is a choice of amino acid residues which may be present at a given position. Importantly, these framework region modifications do not result in a conformational change to the complementarity determining regions as this may adversely alter the binding specificity and/or affinity of the resulting antibody. In certain embodiments, the invention extends to introducing two or more amino acid substitutions to the amino acid residues of the framework regions of the light chain variable region and/or heavy chain variable region.

Accordingly, in certain further embodiments the invention extends to polypeptides, such as an antibody, or antigen binding fragment thereof, which comprise a light chain variable domain having an FR1 region comprising the amino acid sequence of SEQ ID NO:10 which has been modified by one or more of the following amino acid substitutions (where the amino acids are denoted by their single letter code): amino acid residue T at position 5 (T5) is replaced by the amino acid residue M or I; S7 is T; A9 is L or P; S12 is A; L13 is V; S14 is T or R; Q15 is P or R; E16 is D; K18 is E, A, P, T or L; V19 is A; T20 is S; T22 is S or Y and C23 is Y.

In certain further embodiments, the light chain FR2 region having the amino acid sequence of SEQ ID NO:11 may be modified by one or more of the following amino acid substitutions: Y2 is F, I or L; Q3 is R, L or I, Q4 is H, K5 is R, P6 is S or A, G7 is D, A9 is S, T or P, K11 is Q, E or R, L12 is R, P, G, A or 5, I14 is L and Y15 is F, N, S, E or V.

In certain further embodiments, the light chain FR3 region having the amino acid sequence of SEQ ID NO:12 may be modified by one or more of the following amino acid substitutions: G1 is A, V2 is A, P3 is S, S4 is D, F6 is L or V, S7 is I, G8 is A, T13 is A, D14 is E, T16 is S or R, L17 is F, T18 is R or K, S21 is R, G or T, L22 is V, P24 is A, E25 is D, G, I or N, V27 is A, T, G or S, A28 is G and V29 is I or L.

In certain further embodiments, the light chain FR4 region having the amino acid sequence of SEQ ID NO:13 may be modified by one or more of the following amino acid substitutions: G2 is S, Q3 is A, P or T, G4 is E, T5 is P, K6 is Q or S, V7 is L or W, E8 is D or R and I9 is L.

In certain further embodiments, the heavy chain FR1 region having the amino acid sequence of SEQ ID NO:14 may be modified by one or more of the following amino acid substitutions: E1 is D or G, V2 is G, L, E, I or M, Q3 is H, R, A, V, E, K, L, P or S, L4 is V or P, V5 is A, L, E or M, E6 is Q or A, S7 is F, L or T, G9 is E, G10 is D, A, N, E or T, L11 is Q, R, V or W, V12 is A, I or M, Q13 is K, R or N, P14 is F or T, G15 is A, E or T, G16 is E or A, S17 is T or P, L18 is R, R19 is K, T, G or V, L20 is I or V, S21 is Y, A23 is V, L, I or E, A24 is T, V, G, I or S, S25 is P, G or T, G26 is D, R or T and F27 is L, I, S, D, T or V.

In certain further embodiments, the heavy chain FR2 region having the amino acid sequence of SEQ ID NO:15 may be modified by one or more of the following amino acid substitutions: W1 is C, V2 is I, A, F or L, Q4 is L or H, A5 is S, T, G, P, V or D, P6 is L, G7 is E, R or L, K8 is R, E, G, A, M or Q, G9 is E, R, D, T or V, L10 is T, P, F or M, E11 is Q, H, D, L, P or R, W12 is L, C, S, Y, F or M, V13 is L, I or F and S14 is A, T, G or L.

In certain further embodiments, the heavy chain FR3 region having the amino acid sequence of SEQ ID NO:16 may be modified by one or more of the following amino acid substitutions: R1 is Q, F2 is V or L, T3 is A, I or S, 14 is V, L, M or T, S5 is A, F or T, R6 is K, D7 is E or N, N8 is D, T, S, I or G, A9 is G, V, S, D, P or T, K10 is R, E, N, Q, G or M, N11 is D, S, K, H or R, S12 is T, M, I or A, L13 is V, M, A or I, Y14 is F, H, S or T, L15 is I, Q16 is H, E, D, R or A, M17 is L, N18 is D, S, T, H, K, P or R, S19 is G, D, R, N or T, L20 is V, R21 is T, G, K, S or I, A22 is V, D, T, S, G or P, E23 is D, A or V, T25 is A, S or M, A26 is G or V, V27 is M, I, L, F, T, K or Q, Y28 is H, Y29 is F or H, A31 is V, T, G, M, R, S, C or L and K32 is R, S, N, G, A, T, P, D, Q, V, E, I, M.

In certain further embodiments, the heavy chain FR4 region having the amino acid sequence of SEQ ID NO:17 may be modified by one or more of the following amino acid substitutions: W1 is L, G2 is A or S, Q3 is P, H, R or D, T5 is A, S, I or N, L6 is S, Q, P or R, V7 is L, I or P, T8 is F, I, A, S, L, P or Y, V9 is A, S10 is A, C, P or T and S11 is L, A or P.

In certain embodiments, the antibody is a monoclonal antibody. Typically the antibody is a caninised antibody, or a fragment thereof. Alternatively the antibody is a chimeric antibody or a fragment thereof.

In certain further embodiments, the caninised or chimeric TNF neutralising antibody of the invention, or the binding fragment derived therefrom, specifically binds to canine TNF-alpha (tumour necrosis factor alpha) with a binding affinity having an equilibrium dissociation constant (K_{D}) of 1×10⁻⁸ or less. Furthermore, it is preferred that the caninised or chimeric antibodies do not cross-react with any other epitopes present in canines, and further that xenoantibodies are not generated against the antibodies of the invention when they are administered to a canine. Furthermore, it is preferred where short term anti-inflammatory activity is not desired in vivo that the constant domains of the canine TNF-binding antibodies do not mediate any downstream effector functions including, but not limited to, complement fixation and activation, ADCC and Fc receptor binding and activation. Such constant domains are selected from heavy chains consisting of SEQ ID NO:4, aglycosylated SEQ ID NO:4, aglycosylated SEQ ID NO:5, aglycosylated SEQ ID NO:6, aglycosylated SEQ ID NO:7 and SEQ ID NO:7.

In certain yet further embodiments, it is preferred where long-term anti-inflammatory activity is desired that the constant domains of the canine TNF-binding antibodies do mediate effector functions including, but not limited to, complement fixation and activation, ADCC and Fc receptor binding and activation, resulting in the elimination of the canine TNF expressing cells in vivo. Such constant domains are selected from heavy chains consisting of SEQ ID NO:5 and SEQ ID NO:6.

In certain further embodiments, modifications to the amino acid sequence of the constant regions of the heavy chain may be made to the antibodies of the invention. Said modifications may involve the addition, substitution or deletion of one or more amino acid residues. Said amino acid changes are typically performed in order to modify the functional characteristics of the antibody. For example, amino acid modification may be performed to prevent downstream effector functions mediated by the antibody constant domains, for example by preventing the ability of the antibody to bind to Fc receptors, activate complement or induce ADCC. Furthermore, modifications may be made to the amino acid residues of the heavy chain constant domain in order to modify the circulatory half-life of an antibody when it is administered to a canine.

The present invention extends to antibody fragments which bind to canine TNF and sequester its ability to bind to the TNFRs.

In certain embodiments the antibody binding fragment may comprise a heavy chain and light chain sequence of the invention being connected by a flexible linker to form a single chain antibody.

A single chain Fv (scFv) comprises a VH and VL domain. The VH and VL domains associate to form a target binding site. These 2 domains are covalently linked by a peptide linker. A scFv molecule can have the form of VL-linker-VH in cases where the light chain variable domain is required at the N-terminal or the form of VH-linker-VL in cases where the VH domain is required at the N-terminal. Accordingly, in certain further embodiments, the antigen binding fragment is a single chain Fv (scFv) antibody fragment. In certain further embodiments, the antibody binding fragment is selected from the group consisting of, but not limited to, a Fab antibody fragment, a Fab′ antibody fragment, a F(ab′)₂ antibody fragment, a Fv antibody fragment, a scFV antibody fragment and the like.

In some embodiments, the invention provides multispecific or multivalent antibodies comprising an anti-TNF antibody or binding fragment of the invention coupled or conjoined to other antibodies with different binding specificities for use in combination therapy. A multispecific antibody comprises at least one antibody or binding fragment specific to a first TNF epitope, and at least one binding site specific to another epitope present on TNF, or to a different antigen. A multivalent antibody comprises antibodies or antibody binding fragments which have binding specificity to the same TNF epitope. Accordingly, in certain embodiments, the invention extends to an antibody fusion protein comprising four or more Fv regions or Fab regions of the caninised antibodies of the present invention. A yet further embodiment extends to an antibody fusion protein comprising one or more Fab region derived from an antibody described herein along with one or more Fab or Fv regions from antibodies specific for TNF. In certain further embodiments, the invention extends to a bispecific antibody, wherein an antibody or binding fragment thereof according to the present invention is linked to a second antibody or binding fragment thereof which has binding specificity for a secondary target, said target not being TNF. Preferably said secondary target assists in preventing TNF mediated signalling through the TNFR1 receptor. Such multivalent, bispecific or multispecific antibodies can be made by a variety of recombinant methods which would be well known to the person skilled in the art.

A yet further aspect of the invention provides a method for treating or preventing an immune mediated condition in a canine in need thereof, the method comprising the steps of:
providing a therapeutically effective amount of an antibody of the invention, or antigen binding fragment thereof, which specifically binds to canine TNF alpha, and
administering the same to the canine.

In certain embodiments, the antibody is a caninised antibody. In certain embodiments, the caninised antibody comprises a light chain variable domain comprising the amino acid sequence of SEQ ID NO:1 or a sequence which has at least 85% identity thereto and/or a heavy chain variable domain comprising the amino acid sequence of SEQ ID NO:2 or an amino acid sequence having at least 85% sequence homology thereto.

In certain embodiments, the caninised antibody comprises a light chain having the amino acid sequence of SEQ ID NO:3 or a sequence having a sequence identity of at least 85% thereto and/or a heavy chain which comprises, consists of or consists essentially of an amino acid sequence selected from the group consisting of SEQ ID NO:4, SEQ ID NO:5, SEQ ID NO:6 and SEQ ID NO:7 or a sequence having an amino acid identity of at least 85% and more preferably at least 98% identity thereto.

In certain embodiments, the antibody is a chimeric antibody which comprises a light chain having the amino acid sequence of SEQ ID NO:8 and/or a heavy chain having the amino acid sequence of SEQ ID NO:9 or an amino acid sequence having at least 85% sequence homology thereto. In certain embodiments, the chimeric antibody comprises a heavy chain having the amino acid sequence selected from the group consisting of SEQ ID NO:20, SEQ ID NO:21 and SEQ ID NO:22, or an amino acid sequence having at least 85%, 90% or 95% sequence homology thereto.

In certain embodiments, the immune mediated condition is a chronic inflammatory disease. Said chronic inflammatory disease may be selected from the group consisting of, but not limited to, rheumatoid arthritis (RA), osteoarthritis and other polyarthritidies, ankylosing spondylitis (AS), Crohn's disease and ulcerative colitis, psoriasis and psoriatic arthritis (PsA), systemic vasculitis, atopic dermatitis, congestive heart failure (CHF), refractory uveitis, bronchial asthma and allergic conditions. Inflammatory mediated conditions may also include sepsis and shock, diabetes mellitus, and neuro-degenerative conditions such as Alzheimer's disease, Parkinson's disease, stroke and amyotrophic lateral sclerosis. Monoclonal antibodies against TNF-alpha also may also be used for the prevention or treatment of cachexia of cancer, skin tumors such as basal cell carcinoma, colorectal cancers and ovarian cancers.

In certain further embodiments, the immune mediated condition may be a TNF-alpha related disorder or a disorder in which TNF-alpha is a key inflammatory mediator. Such conditions include, but are not limited to, Behcet's disease, bullous dermatitis, neutrophilic dermatitis, toxic epidermal necrolysis, systemic vasculitis, pyoderma gangrenosum, pustular dermatitis, alcoholic hepatitis, cerebral malaria, hemolytic uremic syndrome, pre-eclampsia, allograft rejection, otitis media, snakebite, erythema nodosum, myelodysplastic syndromes, graft versus host disease, dermatomyositis and polymyositis.

According to a yet further aspect of the present invention there is provided a method for the treatment of arthritis or an arthritic condition in a canine subject in need thereof, said method comprising the steps of:
providing a therapeutically effective amount of an anti-canine TNF antibody according to the invention or antigen binding fragment thereof, and
administering the same to the canine.

In certain embodiments, the antibody is a caninised antibody. In certain embodiments, the caninised antibody comprises a light chain variable domain comprising the amino acid sequence of SEQ ID NO:1 or a sequence which has at least 85% identity thereto and/or a heavy chain variable domain comprising the amino acid sequence of SEQ ID NO:2 or an amino acid sequence having at least 85% sequence homology thereto.

In certain embodiments, the arthritis or arthritic condition includes the conditions selected from the group consisting of immune mediated polyarthritis, rheumatoid arthritis, osteoarthritis, psoriatic arthritis, juvenile idiopathic arthritis, ankylosing spondylitis and related conditions.

Typically, the treatment of the arthritis or arthritic condition comprises ameliorating, inhibiting, reducing or suppressing the immune response which is causative of, associated with, or attributable to the arthritic condition.

A further aspect of the present invention provides a method for the treatment of a condition caused by, associated with or resulting in increased expression of canine TNF or increased sensitivity to TNF in a canine in need thereof, said method comprising the steps of:
providing a therapeutically effective amount of an anti-canine TNF antibody according to the invention or antigen binding fragment thereof, and
administering the same to the canine.

In certain embodiments, the foregoing methods of the invention further comprise the step of co-administering at least one further agent which may enhance and/or supplement the effectiveness of the anti-TNF antibody of the invention. For example, the antibody or antigen binding fragment thereof may be co-administered along with one or more additional pharmaceutical compositions, said additional compositions comprising a drug useful for treating a chronic inflammatory condition, in particular a TNF-alpha related disorder. In particular the additional pharmaceutical composition can be methotrexate, soluble p55 or p75 TNF receptor or derivatives thereof, a chimeric or canine antibody to canine TNF, an anti-canine TNF antibody fragment, at least one analgesic, a compound which is a cytokine suppressing anti-inflammatory drug, an NSAID, opioid, corticosteroid, steroid or an antagonist of nerve growth factor, such as an anti-NGF antibody.

Examples of suitable analgesics include, but are not limited to, butorphanol, buprenorphine, fentanyl, flunixin meglumine, merpidine, morphine, nalbuphine and derivatives thereof. Suitable NSAIDS include, but are not limited to, acetaminophen, acetylsalicylic acid, carprofen, etodolac, ketoprofen, meloxicam, firocoxib, robenacoxib, deracoxib and the like.

In certain further embodiments, the at least one further agent may be a therapeutically active agent which may be one or more of the group selected from antibiotic, antifungal, antiprotozoal, antiviral and similar therapeutic agents. Furthermore the at least one further agent may be an inhibitor of mediator(s) of inflammation such as a PGE-receptor antagonist, an immunosuppressive agent, such as cyclosporine, or an anti-inflammatory glucocorticoid. In certain further aspects the at least one further agent may be an agent which is used for the treatment of cognitive dysfunction or impairment, such as memory loss or related conditions which may become increasingly prevalent in older canines. Further still, the at least one further agent may be an anti-hypertensive or other compound used for the treatment of cardiovascular dysfunction, for example to treat hypertension, myocardial ischemia, congestive heart failure and the like. Further still, the at least one further agent may be a diuretic, vasodilator, beta-adrenergic receptor antagonist, angiotensin-II converting enzyme inhibitor, calcium channel blocker or HMG-CoA reductase inhibitor.

In certain embodiments, the antibody or antigen binding fragment is administered to the canine as part of the foregoing methods at a dose ranging from about 0.01 mg/kg of body weight to about 10 mg/kg of body weight, in particular from 0.03 mg/kg of body weight to about 3 mg/kg of body weight.

In various further aspects, the present invention extends to a composition comprising an antibody or binding fragment thereof according to any foregoing aspect of the invention. In certain embodiments, the composition further comprises at least one pharmaceutically acceptable carrier.

A yet further aspect of the invention provides a pharmaceutical composition for treating pain, or a condition resulting in or caused by chronic pain in a canine, comprising a pharmaceutically effective amount of an anti-canine TNF caninised antibody according to the present invention, along with at least one pharmaceutically acceptable carrier, excipient or diluent.

In certain embodiments, the composition may further comprise methotrexate or at least one analgesic, NSAID, opioid, corticosteroid, steroid or antagonist of nerve growth factor.

In various further aspects, the present invention extends to isolated nucleic acid which encodes the antibody or antibody binding fragments of the invention.

Accordingly, a yet further aspect of the invention provides an isolated nucleic acid or polynucleotide that encodes an antibody or antigen binding fragment according to any of the foregoing aspects of the invention. In certain embodiments, the nucleic acid or polynucleotide encodes a light chain variable domain of an anti-canine TNF caninised antibody or antibody fragment having the amino acid sequence of SEQ ID NO:1, or a light chain having the amino acid sequence of SEQ ID NO:3. In certain further embodiments the polynucleotide encodes a heavy chain variable domain of an anti-canine TNF caninised antibody or antibody fragment having the amino acid sequence of SEQ ID NO:2 or a heavy chain having the amino acid sequence selected from the group consisting of SEQ ID NO:4, SEQ ID NO:5, SEQ ID NO:6 and SEQ ID NO:7.

In certain further embodiments the polynucleotide encodes a heavy chain variable domain of an anti-canine TNF chimeric antibody or antibody fragment having the amino acid sequence of SEQ ID NO:8 and/or a heavy chain having the amino acid sequence of SEQ ID NO:9.

In certain embodiments, the isolated nucleic acid further encodes one or more regulatory sequences operably linked thereto. In a further aspect there is provided an expression vector comprising a polynucleotide encoding a heavy and/or light chain variable domain or a heavy and/or light chain constant domain of the invention. In certain embodiments the expression vector further comprises one or more regulatory sequences. In certain embodiments the vector is a plasmid or a retroviral vector. A yet further aspect provides a host cell incorporating the expression vector of the foregoing aspect of the invention. A further aspect of the invention provides a host cell which produces the antibody of any of the foregoing aspects of the invention.

A yet further aspect of the invention provides a method for producing a caninised anti-canine TNF neutralising antibody, the method comprising the step of culturing the host cell of the foregoing aspect of the invention to allow the cell to express the caninised anti-canine TNF neutralising antibody.

A yet further aspect of the present invention provides a method of producing an anti-canine TNF caninised antibody according to the invention comprising the steps of expressing one or more of the polynucleotides/nucleic acids or vectors of the foregoing aspects of the invention which express the light and/or heavy chains of the antibodies of the invention in a suitable host cell, recovering the expressed polypeptides, which may be expressed together in a host cell or separately in different host cells, and isolating antibodies.

A yet further aspect of the invention provides a method for treating, ameliorating or inhibiting pain in a canine, the method comprising the step of administering to the canine an effective amount of a polynucleotide, antibody or fragment according to any of the foregoing aspects of the invention.

A yet further aspect of the invention provides an antibody or antibody binding fragment according to any of the foregoing aspects of the invention, or a pharmaceutical composition according to the foregoing aspects of the invention, or a nucleic acid, or vector comprising the same according to any of the foregoing aspects of the invention for use in the treatment or prevention of pain in a canine.

In certain embodiments the pain is acute pain. In certain further embodiments, the pain is chronic pain. Furthermore, the pain may be post-operative pain or pain resulting from any operating procedure which in canines may include, but is not limited to, orthopaedic surgery, soft tissue surgery, ovariohysterectomy procedures, castration procedures and the like. In certain further embodiments, the pain is chronic pain associated with cancer or a cancerous condition. In certain further embodiments, the pain is associated with, or resulting from, rheumatoid arthritis or osteoarthritis.

A yet further aspect of the invention provides an antibody or antibody binding fragment according to any of the foregoing aspects of the invention, or a pharmaceutical composition according to the foregoing aspects of the invention, or a nucleic acid, or vector comprising the same according to any of the foregoing aspects of the invention for use in the treatment of arthritis, in particular immune mediated polyarthritis, rheumatoid arthritis, osteoarthritis, psoriatic arthritis, juvenile idiopathic arthritis or ankylosing spondylitis.

A yet further aspect of the invention provides use of an antibody or antibody binding fragment according to any of the foregoing aspects of the invention, or a pharmaceutical composition according to the foregoing aspects of the invention, or a nucleic acid, or vector comprising the same according to any of the foregoing aspects of the invention in the preparation of a medicament for the treatment or prevention of a chronic inflammatory disease in a canine.

A yet further aspect of the invention provides use of an antibody or antibody binding fragment according to any of the foregoing aspects of the invention, or a pharmaceutical composition according to the foregoing aspects of the invention, or a nucleic acid, or vector comprising the same according to any of the foregoing aspects of the invention in the preparation of a medicament for the treatment, inhibition amelioration or prevention of rheumatoid arthritis or osteoarthritis in a canine.

In a yet further aspect there is provided a cell line, or a derivative or progeny cell thereof that produces anti-canine TNF neutralising monoclonal antibodies, or fragments thereof according to the invention.

A yet further aspect of the present invention provides a kit for the treatment of chronic inflammatory disease in canines, or for the treatment of a condition associated with pain, or for the treatment, amelioration or inhibition of osteoarthritis or rheumatoid arthritis comprising an anti-canine TNF antibody according to any of the foregoing aspects of the invention and instructions for use of the same.

A yet further aspect of the present invention provides a diagnostic kit for the detection of an anti-canine TNF monoclonal antibody in fluids in vitro, ex vivo and in vivo, for use in determining the concentration of said antibody. The kit may comprise any of the antibodies of the invention or a binding fragment thereof. The kit may comprise instructions for use of same.

### BRIEF DESCRIPTION OF THE FIGURES

FIG. 1 is SEQ ID NO:1 showing the amino acid sequence of a light chain variable domain of a caninised antibody of the invention,

FIG. 2 is SEQ ID NO:2 showing the amino acid sequence of a heavy chain variable domain of a caninised antibody of the invention.

FIG. 3 is SEQ ID NO:3 showing the amino acid sequence of a light chain of a caninised antibody of the invention.

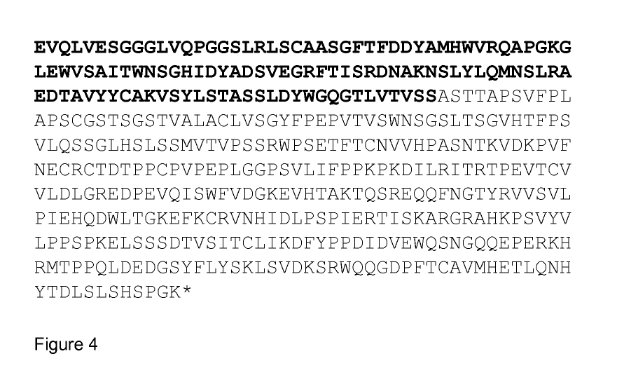

FIG. 4 shows the amino acid sequence (SEQ ID NO:4) of a caninised anti-TNF heavy chain variable domain canine IgG-A heavy chain (caN-HCA). Variable domain residues are shown in bold.

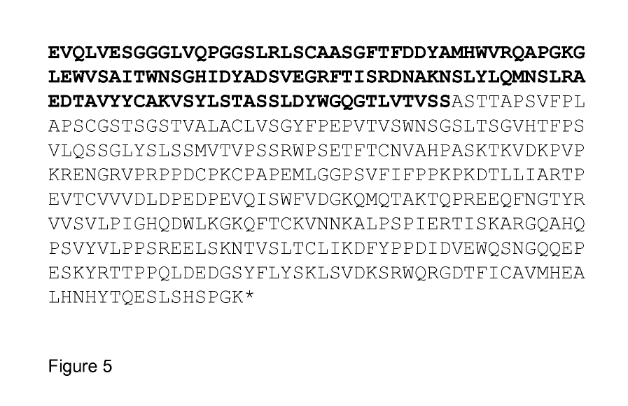

FIG. 5 shows the amino acid sequence (SEQ ID NO:5) of a caninised anti-TNF heavy chain variable domain canine IgG-B heavy chain (caN-HCB). Variable domain residues are shown in bold.

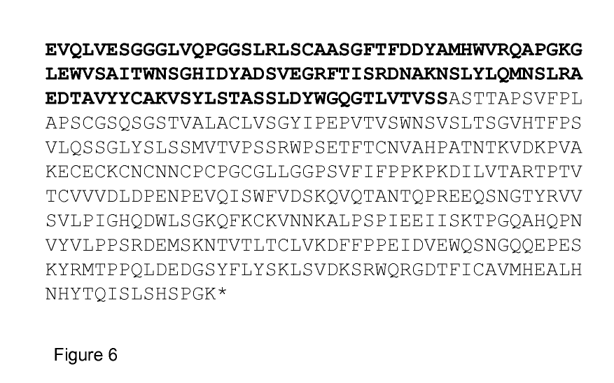

FIG. 6 shows the amino acid sequence (SEQ ID NO:6) of a caninised anti-TNF heavy chain variable domain canine IgG-C heavy chain (caN-HCC). Variable domain residues shown in bold.

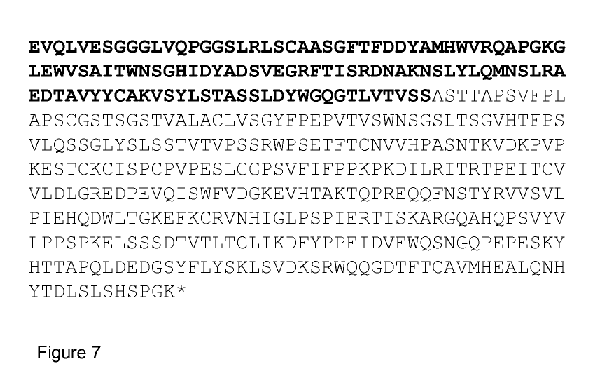

FIG. 7 shows the amino acid sequence (SEQ ID NO:7) of a caninised anti-TNF heavy chain variable domain canine IgG-D heavy chain (caN-HCD). Variable domain residues are shown in bold.

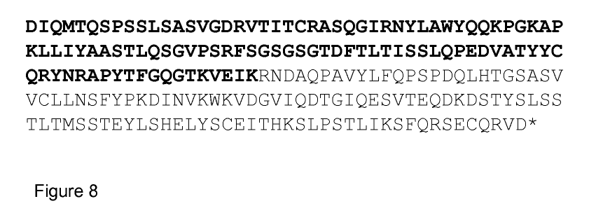

FIG. 8 shows the amino acid sequence (SEQ ID NO:8) of the canine Kappa light chain of a chimeric anti-TNF antibody. Variable domain residues are shown in bold.

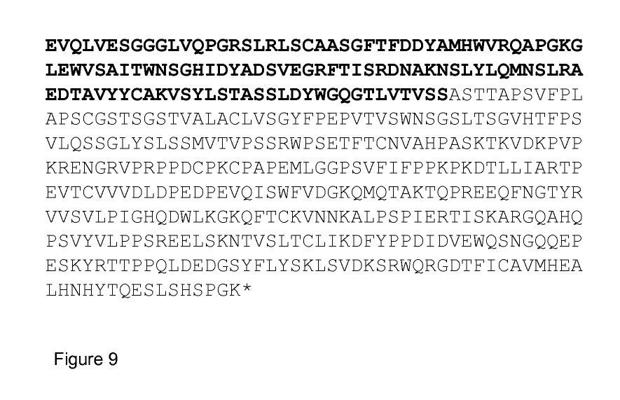

FIG. 9 shows the amino acid sequence (SEQ ID NO:9) of the heavy chain variable domain canine IgG-B heavy chain (caN-HCB) of a chimeric antibody. Variable domain residues are shown in bold.

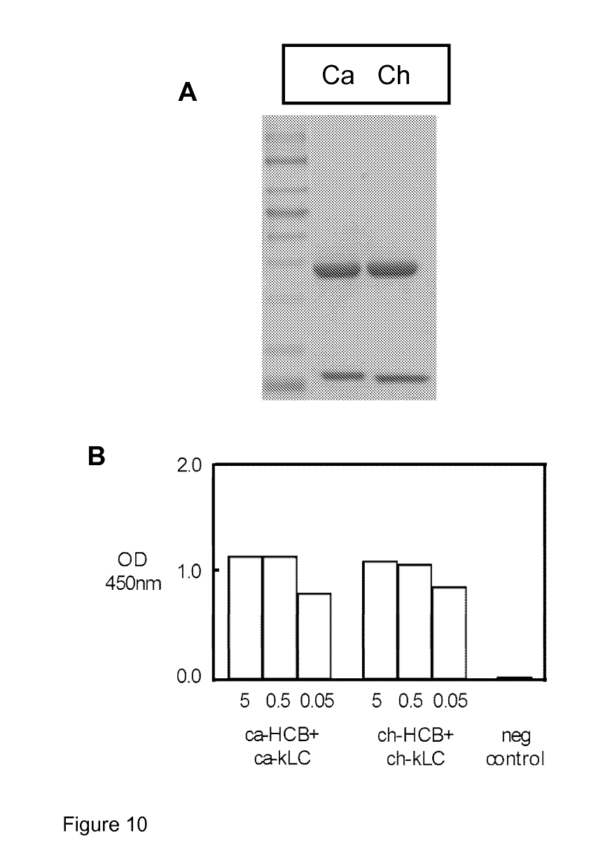

FIG. 10(A) shows the results of co-expressed caninised (SEQ ID NO:3 plus SEQ ID NO:5) and chimeric (SEQ ID NO:8 plus SEQ ID NO:9) antibodies purified using Protein A and analysed by SDS-PAGE, while FIG. 10(B) shows the results of an ELISA showing binding of expressed recombinant proteins to canine TNF-alpha. Results with various dilutions of antibodies from 5 ug/ml to 0.05 ug/ml are shown.

FIG. 11 shows inhibition of canine TNF bioactivity using 293-HEK cells transfected with the NF-kB-EGFP reporter construct pTRH1. These cells respond to canine TNF by fluorescence. Both the caninised (SEQ ID NO:3 plus SEQ ID NO:5) (FIG. 11A) and chimeric (SEQ ID NO:8 plus SEQ ID NO:9) (FIG. 11B) MAbs inhibited TNF-induced fluorescence equally well (quantified in FIG. 11C).

FIG. 12(a) shows a comparison to a further caninised MAb based on anti-TNF MAb clone 148 expressed in CHO cells and purified using Protein A chromatography (Panel A, left lane). The MAb was tested for binding to human TNF (Panel B) and canine TNF (Panel C) in comparison to caninised (Ca) and chimeric (Ch) D2E7 based MAbs from FIG. 10 (background negative control binding is shown by the arrows). As can be seen from this figure, the caninised and chimeric MAbs based on D2E7 and the subject of this invention show unexpectedly strong binding to canine TNF equivalent to that to human TNF whereas caninised MAb 148 shows binding to human TNF alone.

FIG. 12(b) is SEQ ID NO:18 showing the amino acid sequence of a heavy chain of caninised MAb 148.

FIG. 12(c) is SEQ ID NO:19 showing the amino acid sequence of a light chain of caninised MAb 148.

FIG. 13 shows that caninised anti-TNF monoclonal antibodies constructed by co-expression of SEQ ID NO:3 plus SEQ ID NO:5 are able to induce complement mediated lysis of TNF expressing cells.

### DETAILED DESCRIPTION OF THE INVENTION

Following extensive experimentation, the inventor has taken the D2E7 anti-human TNF antibody, an antibody which was not known to bind to canine TNF, and has surprisingly used this as the basis to produce an antagonistic antibody which binds specifically to canine TNF-alpha. The resulting non-immunogenic antibody, which is not produced using standard CDR grafting techniques, is shown to exhibit high affinity binding to canine TNF. The antibody neutralises canine TNF biological function, most specifically by inhibiting the binding of TNF to the cell membrane associated receptor TNFR1. Furthermore, it has also been designed so that the framework and constant regions incorporate only residues present in canine IgG molecules so that when administered to a canine, xenoantibodies are unlikely to be produced there against. Accordingly, the caninised antibody of the invention is suitable for long-term administration for the treatment of chronic inflammatory diseases in canines.

The process of generating the heavy and light chain variable domains for the antibodies of the invention which has been employed by the inventor results in the replacement of specific donor amino acid residues known to be foreign to canines at that position with a canine residue which, based on the inventor's analysis, will retain the conformation of the CDR regions and therefore maintain binding specificity and avidity, while reducing the presence of immunogenic epitopes which may result in neutralising antibodies being generated against the antibody if it were to be administered to canines in an unaltered form. Specifically, the method of preparing antibodies of the invention (known as PETisation) comprises assessing the sequence of the framework regions of a donor (e.g. human) antibody for suitability for administering to a canine by comparing the sequence of the framework regions of the donor antibody with the sequence of an antibody or a pool of antibodies derived from canines. Although the comparison may be between the donor sequence and a single member of the target sequence, it will be obvious that comparison with a pool of target sequences is preferred because this will expand the number of natural options at each Kabat position in the target species. Not only will this increase the chance of a “match” between the donor and the target, but it will also expand the options for replacement where a match does not exist. As a result, a replacement with characteristics as close as possible to the donor will be able to be chosen. Where the donor sequence and the canine sequence differ at any Kabat number or corresponding position, the donor sequence is modified to substitute the amino acid residue in question with an amino acid residue which is known to be natural at that position in canines.

Where substitution of an amino acid residue present in a donor immunoglobulin framework region is required, typically this is undertaken using the principle of conservative substitution wherein an amino acid residue is replaced with an amino acid residue which is natural at that Kabat position in a canine and is as closely related as possible in size, charge and hydrophobicity to the amino acid being substituted in the donor sequence. The intention is to choose a replacement which would cause no, or at least only minimum, perturbation or disruption to the three-dimensional structure of the donor antibody. In certain situations, there will be no clear option and each choice will have benefits and downsides. A final decision may require three-dimensional modelling or even expression of various alternative sequences. However, generally, a clear preference will be available. As a result of this procedure, a change in the donor sequence is only made when that residue would be foreign in the target and the replacement amino acid is as closely related as possible to that which it replaces. Thus, the creation of foreign epitopes is avoided, but the overall three-dimensional structure is preserved and as a result, affinity and specificity are also preserved.

The light and heavy chain constant regions are typically derived from canine (target) derived antibodies. If short term inhibition of TNF is desired, then the heavy chain constant domains are selected or modified such that they do not mediate downstream effector functions, i.e. HCA, HCD or aglycosylated HCB or HCC. If long-term inhibition of TNF is desired, then the heavy chain constant domains are selected or modified such that they mediate downstream effector functions, i.e. HCB or HCC. Furthermore, as the substitution of the framework residues is performed in such a manner that it does not affect the three dimensional conformation of the CDR regions, there will be no variation in binding specificity to the desired target.

There are four major IgG isotypes in man and mouse and while nomenclature is similar they differ in behaviour and function including affinity for bacterial products such as Protein A and Protein G, their ability to activate the complement dependent cytolysis (CDC) and their ability to induce killing of target cells through antibody dependent cellular cytotoxicity (ADCC). The selection of IgG isotypes with CDC and ADCC active or “armed” constant domains is considered to be of clinical benefit when antibodies are designed to eliminate target cells bearing their cognate antigen, such as in oncology, long-term inflammation control or infection control (e.g. in human medical use human IgG1 isotypes are preferred for the above purposes). By contrast, the activation of the immune system is considered undesirable in other settings such as in the relief of inflammation, pain or autoimmunity and so human IgG isotypes with minimal CDC and ADCC activity are preferred (e.g. in such human medical use, IgG4 isotypes are often preferred). Four distinct immunoglobulin gamma (IgG) heavy chain constant domain isotypes have been described in the canine immune system (U.S. Pat. No. 5,852,183, Tang L. et al. 2001. Veterinary Immunology and Immunopathology, 80. 259-270) along with single kappa and lambda constant domain sequences. The four canine heavy chain constant domains A, B, C and D have not been characterised in terms of the functional activity mediated thereby. Despite overall homology to the IgG family, the proteins encoding canine IgG are more related to one another than to family members from other species so it has not been possible by homology alone to define which of the above functions if any can be ascribed to each of the four canine isotypes.

The antibodies of the invention comprise canine derived heavy and light chain constant domains. Furthermore, the complementarity determining regions are derived from the D2E7 antibody. The D2E7 antibody was described by Salfeld et al., 2000 and in U.S. Pat. No. 6,090,382.

The CDR regions derived from the D2E7 antibody are combined with framework region sequences which have been determined by the inventor to preserve CDR tertiary structure, and therefore binding specificity, while preventing xenoantibodies being raised there against, when the antibody is administered to a canine.

Each of the light and heavy chain variable regions contains four framework regions, referred to as FR1-FR4. For each of these framework regions, the inventor has identified a preferred amino acid residue (a so called preferred residue) for each specific position, and furthermore alternative amino acid residues which could also be acceptable at that position. Tables 1 to 8 illustrate the 4 framework regions for each of the heavy and light chains. The tables provide the amino acid position relative to that specific framework region and further according to the Kabat numbering system used to identify the position of a particular residue along the length of the complete heavy or light chain variable domain. The amino acid residues are identified using the single letter system.

TABLE 1
Light chain variable domain FR1 residues

| | Kabat light | | | | |
|---|---|---|---|---|---|
| Light chain | chain | | Alternative | | Human |
| FR1 | numbering | Canine D2E7 | canine | Canine VL | D2E7 |
| position | position | residue | residue(s) | residue set | residue |
| 1 | 1 | D | | D | D |
| 2 | 2 | I | | I | I |
| 3 | 3 | V | | V | Q |
| 4 | 4 | M | | M | M |
| 5 | 5 | T | M, I | M, T, I | T |
| 6 | 6 | Q | | Q | Q |
| 7 | 7 | S | T | T, S | S |
| 8 | 8 | P | | P | P |
| 9 | 9 | A | L, P | L, A, P | S |
| 10 | 10 | S | | S | S |
| 11 | 11 | L | | L | L |
| 12 | 12 | S | A | S, A | S |
| 13 | 13 | L | V | V, L | A |
| 14 | 14 | S | T, R | S, T, R | S |
| 15 | 15 | Q | P, R | P, Q, R | V |
| 16 | 16 | E | D | E, D | G |
| 17 | 17 | E | | E | D |
| 18 | 18 | K | P, T, L, E, A | P, T, L, K, E, A | R |
| 19 | 19 | V | A | A, V | V |
| 20 | 20 | T | S | S, T | T |
| 21 | 21 | I | | I | I |
| 22 | 22 | T | S, Y | S, T, Y | T |
| 23 | 23 | C | Y | C, Y | C |

TABLE 2
Light chain variable domain FR2 residues

| | Kabat light | | | | |
|---|---|---|---|---|---|
| Light chain | chain | | Alternative | | Human |
| FR2 | numbering | Canine D2E7 | canine | Canine VL | D2E7 |
| position | position | residue | residue(s) | residue set | residue |
| 1 | 35 | W | | W | W |
| 2 | 36 | Y | F, I, L | F, Y, I, L | Y |
| 3 | 37 | Q | R, L, I | R, Q, L, I | Q |
| 4 | 38 | Q | H | Q, H | Q |
| 5 | 39 | K | R | K, R | K |
| 6 | 40 | P | S, A | P, S, A | P |
| 7 | 41 | G | D | G, D | G |
| 8 | 42 | Q | | Q | K |
| 9 | 43 | A | S, T, P | S, A, T, P | A |
| 10 | 44 | P | | P | P |
| 11 | 45 | K | Q, E, R | Q, K, E, R | K |
| 12 | 46 | L | R, P, G, A, S | R, L, P, G, A, S | L |
| 13 | 47 | L | | L | L |
| 14 | 48 | I | L | I, L | I |
| 15 | 49 | Y | F, N, S, E, V | Y, F, N, S, E, V | Y |

TABLE 3
Light chain variable domain FR3 residues

| | Kabat light | | | | |
|---|---|---|---|---|---|
| | chain | | | | Human |
| Light chain | numbering | Canine D2E7 | Alternative | Canine VL | D2E7 |
| FR3 position | position | residue | canine residue(s) | residue set | residue |
| 1 | 57 | G | A | G, A | A |
| 2 | 58 | V | A | V, A | V |
| 3 | 59 | P | S | P, S | P |
| 4 | 60 | S | D | D, S | S |
| 5 | 61 | R | | R | R |
| 6 | 62 | F | L, V | F, L, V | F |
| 7 | 63 | S | I | S, I | S |
| 8 | 64 | G | A | G, A | G |
| 9 | 65 | S | | S | S |
| 10 | 66 | G | | G | G |
| 11 | 67 | S | | S | S |
| 12 | 68 | G | | G | G |
| 13 | 69 | T | A | T, A | T |
| 14 | 70 | D | E | D, E | D |
| 15 | 71 | F | | F | F |
| 16 | 72 | T | S, R | T, S, R | T |
| 17 | 73 | L | F | L, F | L |
| 18 | 74 | T | R, K | R, T, K | T |
| 19 | 75 | I | | I | I |
| 20 | 76 | S | | S | S |
| 21 | 77 | S | R, G, T | R, G, S, T | S |
| 22 | 78 | L | V | V, L | L |
| 23 | 79 | E | | E | Q |
| 24 | 80 | P | A | A, P | P |
| 25 | 81 | E | D, G, I, N | D, E, G, I, N | E |
| 26 | 82 | D | | D | D |
| 27 | 82A | V | A, T, G, S | A, V, T, G, S | V |
| 28 | 82B | A | G | G, A | A |
| 29 | 82C | V | I, L | V, I, L | T |
| 30 | 83 | Y | | Y | Y |
| 31 | 84 | Y | | Y | Y |
| 32 | 85 | C | | C | C |

TABLE 4
Light chain variable domain FR4 residues

| | Kabat light | | | | |
|---|---|---|---|---|---|
| Light chain | chain | | | | Human |
| FR4 | numbering | Canine D2E7 | Alternative | Canine VL | D2E7 |
| position | position | residue | canine residue(s) | residue set | residue |
| 1 | 95 | F | | F | F |
| 2 | 96 | G | S | G, S | G |
| 3 | 97 | Q | A, P, T | A, Q, P, T | Q |
| 4 | 98 | G | E | G, E | G |
| 5 | 99 | T | P | T, P | T |
| 6 | 100 | K | Q, S | K, Q, S | K |
| 7 | 101 | V | L, W | V, L, W | V |
| 8 | 102 | E | D, R | D, E, R | E |
| 9 | 103 | I | L | L, I | I |
| 10 | 104 | K | | K | K |

TABLE 5
Heavy chain variable domain FR1 residues

| | Kabat heavy | | | | |
|---|---|---|---|---|---|
| Heavy | chain | Canine | Alternative | | Human |
| chain FR1 | numbering | D2E7 | canine | Canine VH | D2E7 |
| position | position | residue | residue(s) | residue set | residue |
| 1 | 1 | E | D, G | E, D, G | E |
| 2 | 2 | V | G, L, E, I, M | V, G, L, E, I, M | V |
| 3 | 3 | Q | H, R, A, V, E, K, L, | Q, H, R, A, V, E, | Q |
| | | | P, S | K, L, P, S | |
| 4 | 4 | L | V, P | L, V, P | L |
| 5 | 5 | V | A, L, E, M | V, A, L, E, M | V |
| 6 | 6 | E | Q, A | E, Q, A | E |
| 7 | 7 | S | F, L, T | S, F, L, T | S |
| 8 | 8 | G | | G | G |
| 9 | 9 | G | E | G, E | G |
| 10 | 10 | G | D, A, N, E, T | D, A, G, N, E, T | G |
| 11 | 11 | L | Q, R, V, W | L, Q, R, V, W | L |
| 12 | 12 | V | A, I, M | V, A, I, M | V |
| 13 | 13 | Q | K, R, N | K, R, N, Q | Q |
| 14 | 14 | P | F, T | P, F, T | P |
| 15 | 15 | G | A, E, T | G, A, E, T | G |
| 16 | 16 | G | E, A | G, E, A | R |
| 17 | 17 | S | T, P | S, T, P | S |
| 18 | 18 | L | R | L, R | L |
| 19 | 19 | R | K, T, G, V | R, K, T, G, V | R |
| 20 | 20 | L | I, V | L, I, V | L |
| 21 | 21 | S | Y | S, Y | S |
| 22 | 22 | C | | C | C |
| 23 | 23 | A | V, L, I, E | V, L, A, I, E | A |
| 24 | 24 | A | T, V, G, I, S | A, T, V, G, I, S | A |
| 25 | 25 | S | P, G, T | S, P, G, T | S |
| 26 | 26 | G | D, R, T | G, D, R, T | G |
| 27 | 27 | F | L, I, D, S, T, V | F, L, I, D, S, T, V | F |

TABLE 6
Heavy chain variable domain FR2 residues

| | Kabat | | | | |
|---|---|---|---|---|---|
| Heavy | heavy chain | | | | Human |
| Chain FR2 | numbering | Canine | Alternative | Canine VH | D2E7 |
| position | position | D2E7 residue | canine residue(s) | residue set | residue |
| 1 | 36 | W | C | W, C | W |
| 2 | 37 | V | I, A, F, L | V, I, A, F, L | V |
| 3 | 38 | R | | R | R |
| 4 | 39 | Q | L, H | Q, L, H | Q |
| 5 | 40 | A | S, T, G, P, V, G | A, S, T, G, P, V, D | A |
| 6 | 41 | P | L | P, L | P |
| 7 | 42 | G | E, R, L | G, E, R, L | G |
| 8 | 43 | K | R, E, G, A, M, Q | K, R, E, G, A, M, Q | K |
| 9 | 44 | G | E, R, D, T, V | G, E, R, D, T, V | G |
| 10 | 45 | L | P, F, M | L, P, F, M | L |
| 11 | 46 | E | Q, H, D, L, P, R | Q, E, H, D, L, P, R | E |
| 12 | 47 | W | L, C, S, Y, F, M | W, L, C, S, Y, F, M | W |
| 13 | 48 | V | L, I, F | V, L, I, F | V |
| 14 | 49 | S | A, T, G, L | A, T, S, G, L | S |

TABLE 7
Heavy chain variable domain FR3 residues

| | Kabat heavy | | | | |
|---|---|---|---|---|---|
| Heavy | chain | Canine | Alternative | | Human |
| chain FR3 | numbering | D2E7 | canine | Canine VH | D2E7 |
| position | position | residue | residue(s) | residue set | residue |
| 1 | 66 | R | Q | R, Q | R |
| 2 | 67 | F | V, L | F, V, L | F |
| 3 | 68 | T | A, I, S | T, A, I, S | T |
| 4 | 69 | I | V, L, M, T | I, V, L, M, T | I |
| 5 | 70 | S | A, F, T | S, A, F, T | S |
| 6 | 71 | R | K | R, K | R |
| 7 | 72 | D | E, N | D, E, N | D |
| 8 | 73 | N | D, T, S, I, G | N, D, T, S, I, G | N |
| 9 | 74 | A | G, V, S, D, P, T | A, G, V, S, D, P, T | A |
| 10 | 75 | K | R, E, N, Q, G, M | K, R, E, N, Q, G, M | K |
| 11 | 76 | N | D, S, K, H, R | N, D, S, K, H, R | N |
| 12 | 77 | S | T, M, I, A | T, M, I, A, S | S |
| 13 | 78 | L | V, M, A, I | L, V, M, A, I | L |
| 14 | 79 | Y | F, S, H, T | Y, F, S, H, T | Y |
| 15 | 80 | L | I | L, I | L |
| 16 | 81 | Q | H, E, D, R, A | Q, H, E, D, R, A | Q |
| 17 | 82 | M | L | M, L | M |
| 18 | 82A | N | D, S, T, H, K, P, R | N, D, S, T, H, K, P, R | N |
| 19 | 82B | S | G, D, R, N, T | S, G, D, R, N, T | S |
| 20 | 82C | L | V | L, V | L |
| 21 | 83 | R | T, G, K, S, I | R, T, G, K, S, I | R |
| 22 | 84 | A | V, D, T, S, G, P | A, V, D, T, S, G, P | A |
| 23 | 85 | E | D, A, V | E, D, A, V | E |
| 24 | 86 | D | | D | D |
| 25 | 87 | T | A, S, M | T, A, S, M | T |
| 26 | 88 | A | G, V | A, G, V | A |
| 27 | 89 | V | M, I, L, F, T, K, Q | V, M, I, L, F, T, K, Q | V |
| 28 | 90 | Y | H | Y, H | Y |
| 29 | 91 | Y | F, H | Y, F, H | Y |
| 30 | 92 | C | | C | C |
| 31 | 93 | A | V, T, G, M, R, S, C, L | A, V, T, G, M, R, S, | A |
| | | | | C, L | |
| 32 | 94 | K | R, S, N, G, A, T, P, | K, R, S, N, G, A, T, | K |
| | | | D, Q, V, E, I, M | P, D, Q, V, E, I, M | |

TABLE 8
Heavy chain variable domain FR4 residues

| | Kabat heavy | | | | |
|---|---|---|---|---|---|
| Heavy | chain | Canine | Alternative | | Human |
| Chain FR4 | numbering | D2E7 | canine | Canine VH | D2E7 |
| position | position | residue | residue(s) | residue set | residue |
| 1 | 103 | W | L | W, L, | W |
| 2 | 104 | G | A, S | G, A, S | G |
| 3 | 105 | Q | P, H, R, D | Q, P, H, R, D | Q |
| 4 | 106 | G | | G | G |
| 5 | 107 | T | A, S, I, N | T, A, S, I, N | T |
| 6 | 108 | L | S, Q, P, R | L, S, Q, P, R | L |
| 7 | 109 | V | L, I, P | V, L, I, P | V |
| 8 | 110 | T | F, I, A, S, L, P, Y | T, F, I, A, S, L, P, Y | T |
| 9 | 111 | V | A | V, A | V |
| 10 | 112 | S | A, C, P, T | S, A, C, P, T | S |
| 11 | 113 | S | L, A, P | S, L, A, P | S |

The caninised antibody of the invention therefore differs from, for example, a chimeric monoclonal antibody which consists of a complete variable region derived from a first species and constant domains derived from a second species, or from a CDR-grafted caninised antibody, where the complementarity determining regions (CDRs) of the heavy and light chain variable regions comprise amino acid residues derived from a donor antibody and introduced into framework regions (FR) and constant regions (CR) derived from a target antibody or from canine germline sequences.

It is preferred that the caninised antibody substantially retains the binding properties of the parent (donor) antibody from which the CDRs are derived. That means that the caninised antibody will exhibit the same or substantially the same antigen-binding affinity and avidity as the donor antibody from which the CDRs are derived. Ideally, the affinity of the caninised antibody will not be less than 10% of the donor antibody affinity for the target epitope, more preferably not less than about 30%, and most preferably the affinity will be at least that of the parent (donor) antibody. Methods for assaying antigen-binding affinity are well known in the art and include half-maximal binding assays, competition assays, and Scatchard analysis.

As defined hereinbefore, the present invention extends to binding members or antigen binding fragments derived from the caninised antibodies of the invention. Such antigen binding fragments refer to one or more fragments of an antibody that retain the ability to specifically bind to canine TNF. It has been shown that the antigen binding function of an antibody can be performed by fragments of a full length antibody. In certain embodiments, the binding members or antigen binding fragments may be isolated binding members. A binding member or antigen binding fragment of the invention may comprise a fragment of the antibodies of the present invention, e.g. a fragment of a fully caninised antibody molecule, such as the heavy or light chain only, or, for example, the variable domain of the heavy and/or light chain. In certain embodiments, a binding member may typically comprise, consist, or consist essentially of an antibody VH and/or VL domain. VH domains of binding members are also provided as part of the invention. Within each of the VH and VL domains are 3 complementarity determining regions (“CDRs”), along with 4 associated framework regions (“FRs”). A VH domain typically comprises 3 HCDRs (heavy chain complementarity determining regions), and a VL domain typically comprises 3 LCDRs (light chain complementarity regions). Accordingly, a binding member may comprise a VH domain comprising, in sequence, VH CDR1 (or VHCDR1), CDR2 (VHCDR2) and CDR3 (VHCDR3) regions along with a plurality of associated framework regions. A binding member may additionally or alternatively comprise a VL domain comprising VL CDR1, CDR2 and CDR3 domains along with associated framework regions. The VH or VL domains typically comprise four framework regions, FR1, FR2, FR3 and FR4. As used herein, the term “framework region” or “framework sequence” refers to the remaining sequences of a variable region minus the CDRs. Because the exact definition of a CDR sequence can be determined by different naming systems (Kabat, Chothia etc.), the meaning of a framework sequence is subject to correspondingly different interpretations. The six CDRs (VLCDR1, VLCDR2 and VLCDR3 of the light chain and VHCDR1, VHCDR2 and VHCDR3 of the heavy chain) divide the framework regions on the light chain and the heavy chain into four sub-regions known as FR1, FR2, FR3 and FR4 on each chain.

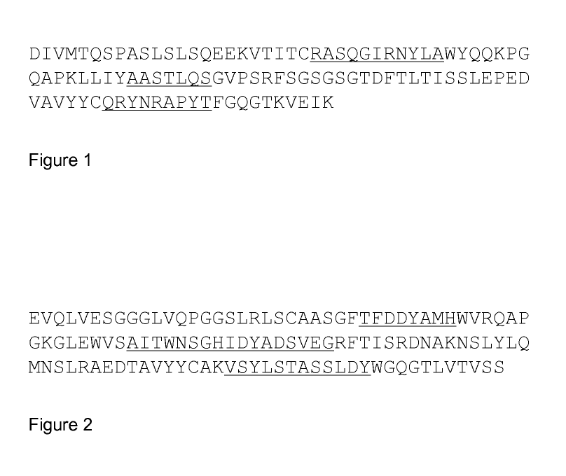

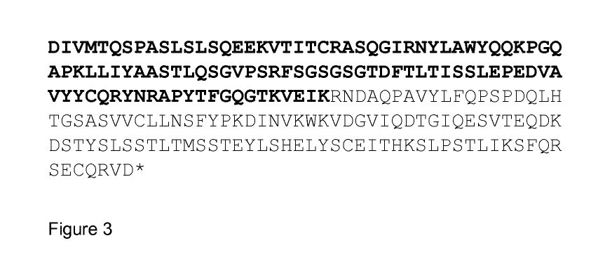

FIG. 1 shows the amino acid sequence of a light chain variable domain of an anti-TNF antibody according to the invention. The CDR1, CDR2 and CDR3 regions are underlined. As such, and as shown in FIG. 1, the VLCDR1 is positioned between FR1 and FR2 framework regions, the VLCDR2 is position between the FR2 and FR3 framework regions and the VLCDR3 is positioned between the FR3 and FR4 framework regions. FIG. 2 shows the amino acid sequence of a heavy chain variable domain of an anti-TNF antibody according to the invention. The CDR1, CDR2 and CDR3 regions are underlined. As with the light chain variable region shown in FIG. 1, the VHCDR1 is positioned between FR1 and FR2 framework regions, the VHCDR2 is position between the FR2 and FR3 framework regions and the VHCDR3 is positioned between the FR3 and FR4 framework regions.

In the tables, the residues of the light chain variable domain and heavy chain variable domain are conventionally numbered according to the numbering system devised by Kabat et al. (Kabat, E. A., Wu, T. T., Perry, H., Gottesman, K. and Foeller, C. (1991) Sequences of Proteins of Immunological Interest, Fifth Edition. NIH Publication No. 91-3242, Kabat et al. (1971) Ann. NY Acad, Sci. 190:382-391). The Kabat numbering system refers to a system of numbering amino acid residues which are more variable (i.e. hypervariable) than other amino acid residues in the heavy and light chain variable regions of an antibody, or an antigen binding portion thereof. The Kabat numbering system is therefore generally used when referring to a residue in the variable domain (approximately residues 1-104 of the light chain and residues 1-113 of the heavy chain). This numbering system may be used in the present specification, where stated. The Kabat residue designations do not always correspond directly with the linear numbering of the amino acid residues of the heavy and light chain variable regions of the present invention provided in the relevant sequences listed herein. In particular, the actual linear amino acid sequence may contain fewer or additional amino acids than in the strict Kabat numbering corresponding to a shortening of, or insertion into, a structural component, whether a framework region or complementarity determining region (CDR), of the basic variable domain structure of the heavy or light chain. The correct Kabat numbering of residues may be determined for any given antibody by alignment of residues in the sequence of the antibody with a standard sequence to which the Kabat numbering has been applied.

As described hereinbefore, an antibody binding fragment may be selected from the group comprising, but not limited to, a Fab fragment, a Fab′ fragment and a scFv (single chain variable fragment), a peptidomimetic, a diabody, or a related multivalent derivative.

In certain embodiments the antibody binding fragment is a Fab or F(ab′)2 fragment, which consists of the VL, VH, CL and CH1 domains of an antibody. In certain embodiments, the VL domain has an amino acid sequence of SEQ ID NO:1, and the VH domain has an amino acid sequence of SEQ ID NO:2. In certain embodiments, the CL and CH1 domains are based on the amino acid sequence of a CL and CH1 domain of a canine immunoglobulin.

Techniques used for the recombinant production of Fab, Fab′ and F(ab′)2 fragments are well known to the person skilled in the art and include those disclosed in International PCT Patent Publication WO 92/22324, and in Sawai et al., “Direct Production of the Fab Fragment Derived From the Sperm Immobilizing Antibody Using Polymerase Chain Reaction and cDNA Expression Vectors”, 1995, AJRI 34:26-34. Examples of techniques which can be used to produce scFv (single chain Fv fragments) are disclosed in Huston et al., “Protein Engineering of Single-Chain Fv Analogs and Fusion Proteins”, Methods in Enzymology, vol. 203:46-88 (1991), the contents of which are incorporated by reference.

In certain embodiments, antibody fragments can be derived from full length antibodies by proteolytic digestion according to the method of Morimoto (Morimoto et al., “Single-step purification of F(ab′)2 fragments of mouse monoclonal antibodies (immunoglobulins G1) by hydrophobic interaction high performance liquid chromatography using TSKgel Phenyl-5PW” Journal of Biochemical and Biophysical Methods 24:107-117 (1992)). Antibody fragments can also be produced directly by host cells (Carter et al., “High level Escherichia coli expression and production of a bivalent humanized antibody fragment” Bio/Technology 10:163-167 (1992)).

In addition to providing a caninised monoclonal antibody which has binding specificity to canine TNF and which antagonises canine TNF function, the present invention further extends to binding members other than antibodies comprising a pair of binding domains based on the amino acid sequence of a VL (light chain variable) region as defined in SEQ ID NO:1 and an amino acid sequence of a VH (heavy chain variable) region as defined in SEQ ID NO:2. In particular, the invention extends to single binding domains which are based on either the VL or VH region of the caninised antibodies of the antibodies of the invention.

Accordingly, in certain further embodiments of the present invention, there is provided a binding member comprising, consisting or consisting essentially of a single binding domain derived from the caninised antibody of the invention. In certain embodiments, the single binding domain is derived from the amino acid sequence of the VH (heavy chain variable domain) as defined in SEQ ID NO:2. Such a binding domain may be used as a targeting agent to canine TNF.

In certain embodiments, further engineering techniques can be used to modify the antibodies of the present invention, for example by including modifications of the Fc region which can alter serum half-life, complement fixation, Fc receptor binding and/or antigen dependent cellular cytotoxicity. Further, in certain embodiments, antibodies or antibody fragments can be produced which have altered glycosylation patterns. In certain embodiments, an antibody of the invention is altered to increase or decrease the extent to which the antibody is glycosylated. Glycosylation of polypeptides is typically either N-linked or O-linked. N-linked refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

In certain further embodiments, the anti-canine TNF antibodies of the invention can be PEGylated by reacting the antibody with a polyethylene glycol (PEG) derivative. In certain embodiments, the antibody is defucosylated and therefore lacks fucose residues.

In certain embodiments, modifications in the biological properties of an antibody may be accomplished by selecting substitutions that affect (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (A. L. Lehninger, in Biochemistry, 2^{nd} Ed., 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic:

Lys (K), Arg (R), His (H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Such substituted residues also may be introduced into the conservative substitution sites or, into the remaining (e.g., non-conserved) sites.

In various further aspects, the present invention extends to an immunoconjugate comprising an anti-canine TNF antibody of the invention, or an antigen binding portion thereof linked to a partner molecule. In certain embodiments, such an antibody-partner molecule conjugate is conjugated by means of a chemical linker, such as a peptidyl linker, a hydrazine linker or a disulphide linker. In certain embodiments, the coupling partner is an effector molecule, label, drug, or carrier molecule. Suitable techniques for coupling the antibodies of the invention to both peptidyl and non-peptidyl coupling partners will be well known to persons skilled in the art. Examples of suitable labels include detectable labels, such as a radiolabel, or an enzymatic label, such as horse radish peroxidase, or chemical moieties, such as biotin. Alternatively, the label may be a functional label, for example, ricin, or pro-drugs which are capable of converting prodrugs into active drugs at the site of antibody binding.

In various further aspects, the present invention extends to polynucleotides, and in particular isolated polynucleotides, which encode the caninised antibodies, antibody fragments and binding members of the present invention. As defined herein, a “polynucleotide” includes any polyribonucleotide or polydeoxyribonucleotide, which may be unmodified RNA or DNA, or modified RNA or DNA, including without limitation, single and double stranded RNA, and RNA which is a mixture of single and double stranded regions. A polynucleotide of the invention, e.g. a polynucleotide which encodes a polypeptide or polypeptides of the invention includes allelic variants thereof and/or their complements including a polynucleotide that hybridises to such nucleotide sequences under conditions of moderate or high stringency.

The present invention further extends to antibody mimetics, such as domain antibodies, nanobodies, unibodies, versabodies, and duocalins which are based on the canine TNF antibodies of the present invention. A wide variety of antibody mimetic technologies are known to the person skilled in the art. For example, so called, domain antibodies (Domantis, UK) are small functional binding units of antibodies which correspond to the variable regions of either the light or heavy chains of human antibodies. Directions for the production of such domain antibodies can be found in U.S. Pat. No. 6,291,158, U.S. Pat. No. 6,582,915 and U.S. Pat. No. 6,593,081. Nanobodies are antibody-derived therapeutic proteins which contain unique structural and functional properties of naturally occurring heavy chain antibodies found in camelids. Unibodies are a further antibody fragment technology, based upon the removal of the hinge region of IgG4 antibodies. The deletion of the hinge region results in a molecule which is approximately half the size of a traditional IgG4 antibody and which has a univalent binding region. Unibodies preserve the property of IgG4 antibodies of being inert and therefore not inducing immune responses.

### Antibody Production

The antibodies and binding members of the invention may be produced wholly or partly by chemical synthesis. For example, the antibodies and binding members of the invention can be prepared by techniques which are well known to the person skilled in the art, such as standard liquid peptide synthesis, or by solid-phase peptide synthesis methods. Alternatively, the antibodies and binding members may be prepared in solution using liquid phase peptide synthesis techniques, or further by a combination of solid-phase, liquid phase and solution chemistry.

The present invention further extends to the production of the antibodies or binding members of the invention by expression of a nucleic acid which encodes at least one amino acid which comprises an antibody of the invention in a suitable expression system, such that a desired peptide or polypeptide can be encoded. For example, a nucleic acid encoding the amino acid light chain and a second nucleic acid encoding an amino acid heavy chain can be expressed to provide an antibody of the present invention.

Accordingly, in certain further aspects of the invention, there is provided nucleic acids encoding amino acid sequences which form the antibodies or binding members of the present invention.

Typically, nucleic acids encoding the amino acid sequences which form antibodies or binding members of the present invention can be provided in an isolated or purified form, or provided in a form which is substantially free of material which can be naturally associated with it, with the exception of one or more regulatory sequences. Nucleic acids which expresses an antibody or binding member of the invention may be wholly or partially synthetic and may include, but is not limited to DNA, cDNA and RNA.

Nucleic acid sequences encoding the antibodies or binding members of the invention can be readily prepared by the skilled person using techniques which are well known to those skilled in the art, such as those described in Sambrook et al. “Molecular Cloning”, A laboratory manual, cold Spring Harbor Laboratory Press, Volumes 1-3, 2001 (ISBN-0879695773), and Ausubel et al. Short Protocols in Molecular Biology. John Wiley and Sons, 4th Edition, 1999 (ISBN-0471250929). Said techniques include (i) the use of the polymerase chain reaction (PCR) to amplify samples of nucleic acid, (ii) chemical synthesis, or (iii) preparation of cDNA sequences. DNA encoding antibodies or binding members of the invention may be generated and used in any suitable way known to those skilled in the art, including taking encoding DNA, identifying suitable restriction enzyme recognition sites either side of the portion to be expressed, and cutting out said portion from the DNA. The excised portion may then be operably linked to a suitable promoter and expressed in a suitable expression system, such as a commercially available expression system. Alternatively, the relevant portions of DNA can be amplified by using suitable PCR primers. Modifications to the DNA sequences can be made by using site directed mutagenesis.

Nucleic acid sequences encoding the antibodies or binding members of the invention may be provided as constructs in the form of a plasmid, vector, transcription or expression cassette which comprises at least one nucleic acid as described above. The construct may be comprised within a recombinant host cell which comprises one or more constructs as above. Expression may conveniently be achieved by culturing, under appropriate conditions, recombinant host cells containing suitable nucleic acid sequences. Following expression, the antibody or antibody fragments may be isolated and/or purified using any suitable technique, then used as appropriate.

Systems for cloning and expression of a polypeptide in a variety of different host cells are well known. Suitable host cells include bacteria, mammalian cells, yeast, insect and baculovirus systems. Mammalian cell lines available in the art for expression of a heterologous polypeptide include Chinese hamster ovary (CHO) cells, HeLa cells, baby hamster kidney cells and NS0 mouse myeloma cells. A common, preferred bacterial host is E. coli. The expression of antibodies and antibody fragments in prokaryotic cells such as E. coli is well established in the art. Expression in eukaryotic cells in culture is also available to those skilled in the art as an option for production of a binding member.

General techniques for the production of antibodies are well known to the person skilled in the field, with such methods being discussed in, for example, Kohler and Milstein (1975) Nature 256: 495-497; U.S. Pat. No. 4,376,110; Harlow and Lane, Antibodies: a Laboratory Manual, (1988) Cold Spring Harbor. Techniques for the preparation of recombinant antibody molecules are described in the above references and also in, for example, European Patent Number 0,368,684.

In certain embodiments of the invention, recombinant nucleic acids comprising an insert coding for a heavy chain variable domain and/or for a light chain variable domain of antibodies or binding members are employed. By definition, such nucleic acids comprise single stranded nucleic acids, double stranded nucleic acids consisting of said coding nucleic acids and of complementary nucleic acids thereto, or these complementary (single stranded) nucleic acids themselves.

Furthermore, nucleic acids encoding a heavy chain variable domain and/or a light chain variable domain of antibodies can be enzymatically or chemically synthesised nucleic acids having the authentic sequence coding for a naturally-occurring heavy chain variable domain and/or for the light chain variable domain, or a mutant thereof.

An antibody of the invention may be produced by recombinant means, not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process a native antibody signal sequence, the signal sequence is substituted by a prokaryotic signal sequence selected, for example, from the group of the alkaline phosphatase, penicillinase, lpp, or heat-stable enterotoxin II leaders.

The term “isolated”, when used in reference to the caninised antibodies of the invention, or to binding members derived therefrom, or polynucleotides which encode the same, refers to the state in which said antibodies, binding members or nucleic acids (polynucleotides) are provided in an isolated and/or purified form, that is they have been separated, isolated or purified from their natural environment, and are provided in a substantially pure or homogeneous form, or, in the case of nucleic acid, free or substantially free of nucleic acid or genes of origin other than the sequence encoding a polypeptide with the required function. Accordingly, such isolated antibodies, binding members and isolated nucleic acids will be free or substantially free of material with which they are naturally associated, such as other polypeptides or nucleic acids with which they are found in their natural environment, or the environment in which they are prepared (e.g. cell culture) when such preparation is by recombinant DNA technology practised in vitro or in vivo.

Antibodies, binding members and nucleic acids may be formulated with diluents or adjuvants and still, for practical purposes, be considered as being provided in an isolated form. For example the antibodies and binding members can be mixed with gelatin or other carriers if used to coat microtitre plates for use in immunoassays, or will be mixed with pharmaceutically acceptable carriers or diluents when used in diagnosis or therapy. The antibodies or binding members may be glycosylated, either naturally or by systems of heterologous eukaryotic cells (e.g. CHO or NSO cells, or they may be (for example if produced by expression in a prokaryotic cell) unglycosylated.

Heterogeneous preparations comprising anti-canine TNF caninised antibody molecules also form part of the invention. For example, such preparations may be mixtures of antibodies with full-length heavy chains and heavy chains lacking the C-terminal lysine, with various degrees of glycosylation and/or with derivatized amino acids, such as cyclization of an N-terminal glutamic acid to form a pyroglutamic acid residue.

### Pharmaceutical Compositions

Typically the pharmaceutical compositions of the invention are formulated in a liquid formulation, a lyophilized formulation, a lyophilized formulation that is reconstituted as a liquid, or as an aerosol formulation. In certain embodiments, the antibody in the formulation is at a concentration of: about 0.5 mg/ml to about 250 mg/ml, about 0.5 mg/ml to about 45 mg/ml, about 0.5 mg/ml to about 100 mg/ml, about 100 mg/ml to about 200 mg/ml, or about 50 mg/ml to about 250 mg/ml.

In certain embodiments, the formulation further comprises a buffer. Typically the pH of the formulation is from about pH 5.5 to about pH 6.5. In certain embodiments, the buffer may comprise from about 4 mM to about 60 mM histidine buffer, about 5 mM to about 25 mM succinate buffer, or about 5 mM to 25 mM acetate buffer. In certain embodiments, the buffer comprises sodium chloride at a concentration of from about 10 mM to 300 mM, typically at around 125 mM concentration and sodium citrate at a concentration of from about 5 mM to 50 mM, typically 25 mM. In certain embodiments the formulation can further comprise a surfactant at a concentration of just above 0% to about 0.2%. In certain embodiments the surfactant is selected from the group consisting of, but not limited to, polysorbate-20, polysorbate-40, polysorbate-60, polysorbate-65, polysorbate-80, polysorbate-85, and combinations thereof. In a preferred embodiment, the surfactant is polysorbate-20 and may further comprise sodium chloride at a concentration of about 125 mM and sodium citrate at a concentration of about 25 mM.

### Administration

The antibodies or binding members of the present invention may be administered alone, but will preferably be administered as a pharmaceutical composition which will generally comprise a suitable pharmaceutically acceptable excipient, diluent or carrier selected depending on the intended route of administration. Examples of suitable pharmaceutical carriers include; water, glycerol, ethanol and the like.

The monoclonal antibody or binding member of the present invention may be administered to a canine patient in need of treatment via any suitable route. Typically, the composition can be administered parenterally by injection or infusion. Examples of preferred routes for parenteral administration include, but are not limited to, intravenous, intracardial, intraarterial, intraperitoneal, intraarticular, intramuscular, intracavity, subcutaneous, transmucosal, or transdermal. Routes of administration may further include topical and enteral, for example, mucosal (including pulmonary), oral, nasal or rectal.

In embodiments where the composition is delivered as an injectable composition, for example in intravenous, intradermal or subcutaneous application, the active ingredient can be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection or, Lactated Ringer's injection. Preservatives, stabilisers, buffers, antioxidants and/or other additives may be included, as required.

The composition may also be administered via microspheres, liposomes, other microparticulate delivery systems or sustained release formulations placed in certain tissues including blood.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition ISBN 0-912734-04-3 and Pharmaceutical Dosage Forms and Drug Delivery Systems; Ansel, H. C. et al. 7th Edition ISBN 0-683305-72-7, the entire disclosures of which is herein incorporated by reference.

The antibodies and compositions of the invention are typically administered to a subject in a “therapeutically effective amount”, this being an amount sufficient to show benefit to the subject to whom the composition is administered. The actual dose administered, and rate and time-course of administration, will depend on, and can be determined with due reference to, the nature and severity of the condition which is being treated, as well as factors such as the age, sex and weight of the subject being treated, as well as the route of administration. Further due consideration should be given to the properties of the composition, for example, its binding activity and in-vivo plasma life, the concentration of the antibody or binding member in the formulation, as well as the route, site and rate of delivery.

Dosage regimens can include a single administration of the antibody or composition of the invention, or multiple administrative doses of the antibody or composition. The antibody or antibody containing compositions can further be administered sequentially or separately with other therapeutics and medicaments which are used for the treatment of the condition for which the antibody or binding member of the present invention is being administered to treat.

Examples of dosage regimens which can be administered to a subject can be selected from the group comprising, but not limited to, 1 μg/kg/day through to 20 mg/kg/day, 1 μg/kg/day through to 10 mg/kg/day, 10 μg/kg/day through to 1 mg/kg/day. In certain embodiments, the dosage will be such that a plasma concentration of from 1 μg/ml to 100 μg/ml of the antibody is obtained. However, the actual dose of the composition administered, and rate and time-course of administration, will depend on the nature and severity of the condition being treated. Prescription of treatment, e.g. decisions on dosage etc, is ultimately within the responsibility and at the discretion of veterinary practitioners and other veterinary doctors, and typically takes account of the disorder to be treated, the condition of the individual patient, the site of delivery, the method of administration and other factors known to practitioners.

### DEFINITIONS

Unless otherwise defined, all technical and scientific terms used herein have the meaning commonly understood by a person who is skilled in the art in the field of the present invention. The meaning and scope of the terms should be clear, however, in the event of any ambiguity, definitions provided herein take precedent over any dictionary or extrinsic definition.

Throughout the specification, unless the context demands otherwise, the terms “comprise” or “include”, or variations such as “comprises” or “comprising”, “includes” or “including” will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well as two or more different active agents in combination, while references to “a carrier” includes mixtures of two or more carriers as well as a single carrier, and the like. Further, unless otherwise required by context, singular terms shall include pluralities and plural terms shall include the singular.

As defined herein, the term TNF neutralising antibody or similar describes an antibody that is capable of neutralising the biological activity and signalling of TNF. The neutralising antibody, which may also be referred to as an antagonistic antibody, or a blocking antibody, specifically and preferably selectively, binds to TNF and inhibits one or more biological activities of TNF. For example, the neutralising antibody may inhibit the binding of a TNF to its target ligand, such as the cell membrane bound TNF Receptor 1 (TNFR1) receptor (CD120a).

As used herein, the term “biological activity” refers to any one or more inherent biological properties of a molecule (whether present naturally as found in vivo, or provided or enabled by recombinant means). Biological properties include, but are not limited to, receptor binding and/or activation, induction of cell signalling or cell proliferation, inhibiting cell growth, induction of cytokine production, induction of apoptosis and enzymatic activity.

The term “complementarity determining region (CDR)”, as used herein, refers to amino acid sequences which together define the binding affinity and specificity of the natural Fv region of a native immunoglobulin binding site as delineated by Kabat et al. (Kabat, E. A., Wu, T. T., Perry, H., Gottesman, K. and Foeller, C. (1991) Sequences of Proteins of Immunological Interest, Fifth Edition. NIH Publication No. 91-3242). The term framework region (FR), as used herein, refers to amino acid sequences interposed between CDRs. These portions of the antibody serve to hold the CDRs in appropriate orientation (allows for CDRs to bind antigen).

The term “constant region (CR)” as used herein, refers to the portion of the antibody molecule which confers effector functions. In the present invention, constant regions typically mean canine constant regions, that is that the constant regions of the subject canininsed antibodies are derived from canine immunoglobulins. The heavy chain constant region can be selected from any of the four isotypes: A, B, C or D.

The term “chimeric antibody” as used herein refers to an antibody containing sequences derived from two different antibodies, which are derived from different species. Most typically chimeric antibodies comprise variable domains derived from a donor specifies which bind specifically to a target epitope and constant domains derived from antibodies obtained from the target species to whom the antibody is to be administered.

The term “immunogenicity” as used herein refers to a measure of the ability of a targeting protein or therapeutic moiety to elicit an immune response (humoral or cellular) when administered to a recipient. The present invention is concerned with the immunogenicity of the subject caninised antibodies. Preferably the antibodies of the present invention have no immunogenicity, that is that no xenoantibodies will be raised against them when administered to a canine.

The term “identity” or “sequence identity” as used herein, means that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. This may be referred to as conservative substitution. Preferably when the amino acid sequences of the invention are modified by way of conservative substitution of any of the amino acid residues contained therein, these changes have no effect on the binding specificity or functional activity of the resulting antibody when compared to the unmodified antibody.

Sequence identity with respect to a (native) polypeptide of the invention and its functional derivative relates to the percentage of amino acid residues in the candidate sequence which are identical with the residues of the corresponding native polypeptide, after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology. Methods and computer programs for performing an alignment of two or more amino acid sequences and determining their sequence identity or homology are well known to the person skilled in the art. For example, the percentage of identity or similarity of 2 amino acid sequences can be readily calculated using algorithms e.g. BLAST (Altschul et al. 1990), FASTA (Pearson & Lipman 1988), or the Smith-Waterman algorithm (Smith & Waterman 1981).

As used herein, reference to an amino acid residue having the “highest homology” to a second amino acid residue refers to the amino acid residue which has the most characteristics or properties in common with the second amino acid residue. In determining whether an amino acid residue has the highest homology to a second amino acid residue, an assessment may typically be made of factors such as, but not limited to, charge, polarity, hydrophobicity, side arm mass and side arm dimension.

The term “corresponding position” as used herein to refer to an amino acid residue that is present in a second sequence at a position corresponding to a specified amino acid residue in a first sequence is intended to refer to the position in the second sequence which is the same position as the position in the first sequence when the two sequences are aligned to allow for maximum sequence identity between the two sequences. Amino acid residues at corresponding positions have the same Kabat numbering.

The term “consists essentially of” or “consisting essentially of” as used herein means that a polypeptide may have additional features or elements beyond those described provided that such additional features or elements do not materially affect the ability of the antibody or antibody fragment to have binding specificity to canine TNF. That is, the antibody or antibody fragments comprising the polypeptides may have additional features or elements that do not interfere with the ability of the antibody or antibody fragments to bind to canine TNF and antagonise canine TNF functional activity. Such modifications may be introduced into the amino acid sequence in order to reduce the immunogenicity of the antibody. For example, a polypeptide consisting essentially of a specified sequence may contain one, two, three, four, five or more additional, deleted or substituted amino acids, at either end or at both ends of the sequence provided that these amino acids do not interfere with, inhibit, block or interrupt the role of the antibody or fragment in binding to canine TNF and sequestering its biological function. Similarly, a polypeptide molecule which contributes to the canine TNF antagonistic antibodies of the invention may be chemically modified with one or more functional groups provided that such functional groups do not interfere with the ability of the antibody or antibody fragment to bind to canine TNF and antagonise its function.

As used herein, the term “effective amount” or “therapeutically effective amount” means the amount of an agent, binding compound, small molecule, fusion protein or peptidomimetic of the invention which is required to suppress canine TNF binding to the TNFR1 receptor.

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide.

As herein defined an “antibody” encompasses antigen-binding proteins which specifically bind to a target antigen of interest, in this case canine tumour necrosis factor, having one or more polypeptides that can be recombinantly prepared or which are genetically encodable by immunoglobulin genes, or fragments of immunoglobulin genes. The term “antibody” encompasses monoclonal and chimeric antibodies, in particular caninised antibodies, and further encompasses polyclonal antibodies or antibodies of any class or subtype. An “antibody” further extends to hybrid antibodies, bispecific antibodies, heteroantibodies and to functional fragments thereof which retain antigen binding.

The phrase “specifically binds to” refers to the binding of an antibody to a specific protein or target which is present amongst a heterogeneous population of proteins. Hence, when present in specific immunoassay conditions, the antibodies bind to a particular protein, in this case canine TNF, and do not bind in a significant amount to other proteins present in the sample.

As defined herein, a “canine” may also be referred to as a dog. Canines can be categorised as belonging to the subspecies with the trinomial name Canis lupus familiaris (Canis familiaris domesticus) or Canis lupus dingo. Canines include any species of dog and includes both feral and pet varieties, the latter also being referred to as companion animals.

As defined herein, the term “xenoantibody” refers to an antibody which is raised by the host against an epitope which is foreign to the host.

The present invention will now be described with reference to the following examples which are provided for the purpose of illustration and are not intended to be construed as being limiting on the present invention. The methods and techniques of the present invention are generally performed according to conventional methods well known in the art and as described in various general and more specific references that are cited and discussed throughout the present specification unless otherwise indicated.

### EXAMPLES

### Example 1

### Expression of DNA Encoding Canine PETised Anti-Canine TNF MAbs

The sequences of SEQ ID NO:1 to SEQ ID NO:9 were designed and built as DNAs using oligonucleotide synthesis and subcloned to pcDNA3.1+ expression vectors and transfected in various combinations into CHO cells.

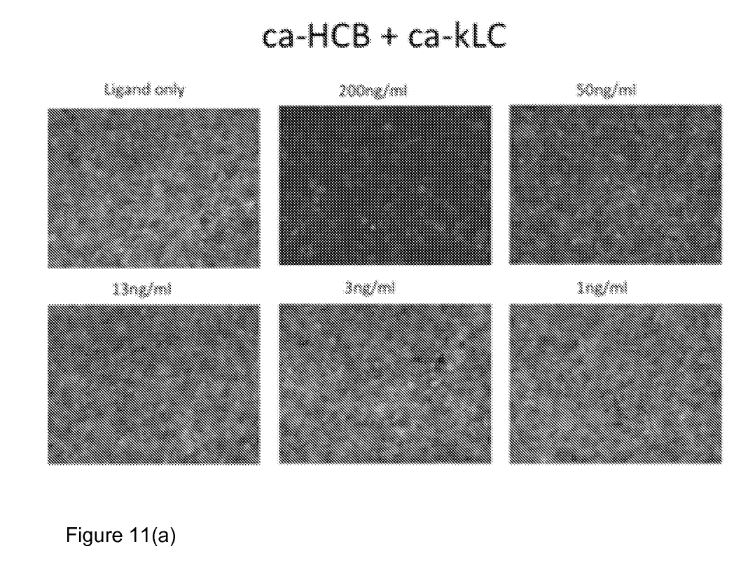

cDNAs encoding caninised anti-TNF monoclonal antibodies having the amino acid sequence of SEQ ID NO:3 (light chain) and SEQ ID NO:5 (heavy chain) and a chimeric anti-TNF monoclonal antibody having a light chain with the amino acid of SEQ ID NO:8 and a heavy chain of SEQ ID NO:9 were subcloned into pcDNA3.1+ (Invitrogen/Life technologies). CHO cells were co-transfected with combinations of either caninised heavy and light chain sequences of SEQ ID NO:3 and SEQ ID NO:5 (ca-HCB+ca-kLC) or chimeric heavy and light chains of SEQ ID NO:8 and SEQ ID NO:9 (ch-HCB+ch-kLC). The resultant supernatants were purified on Protein A, analysed by SDS-PAGE (FIG. 10A) and tested for binding to canine TNFa (coated at 5 ug/ml; R&D systems) at the indicated concentrations of antibody (ug/ml) by ELISA and detected using anti-canine polyclonal antibody-horseradish peroxidase conjugate (Sigma A9042) (FIG. 10B). The negative control was the detection polyclonal on coated antigen alone.

### Example 2

### Inhibition of Canine TNF Activity

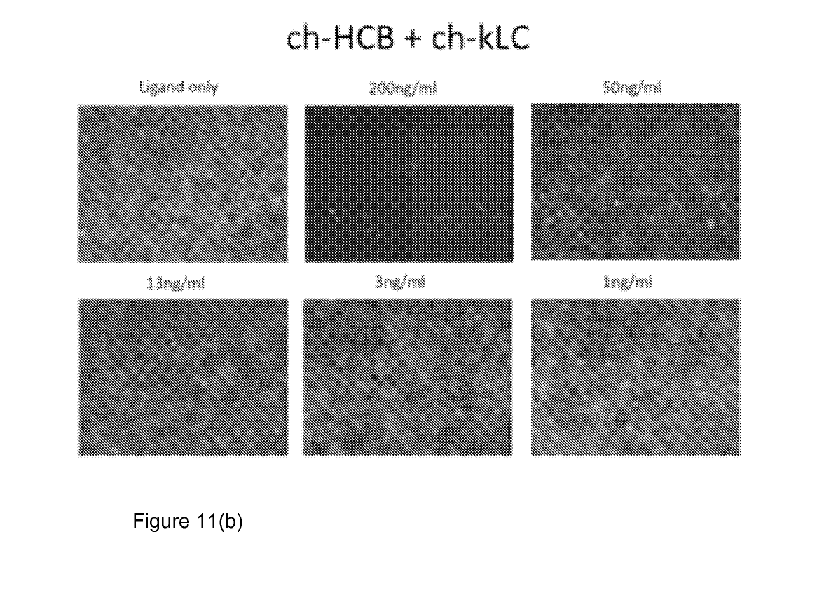

Purified antibodies were tested for their ability to inhibit canine TNF activity using 293-HEK cells transfected with pTRH1 to produce a TNF sensitive NF-kB-EGFP reporter cell line that responds to human TNF by fluorescence (Vince et al, Cell 131, 682, 2007). It was first demonstrated that canine TNF activates GFP expression in these cells and then the canine antibodies shown in FIG. 11 were tested for their ability to inhibit 1 ng/mL canine TNF.

As shown in FIG. 11, both the caninised antibody produced by co-transfection of SEQ ID NO:3 and SEQ ID NO:5 (ca-HCB+ca-kLC) and the chimeric antibody produced by transfection of SEQ ID NO:8 and SEQ ID NO:9 (ch-HCB+ch-kLC) are potent inhibitors of canine TNF in this assay.

Together these results show that the caninised antibodies of the invention and the human-canine chimera construct bind canine TNF and are equipotent by both ELISA and inhibition assay, demonstrating that the caninisation process has produced a fully active canine version of the original D2E7 antibody.

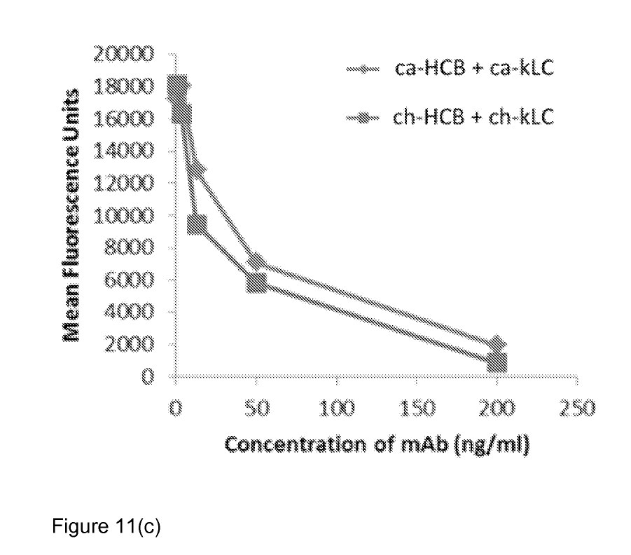

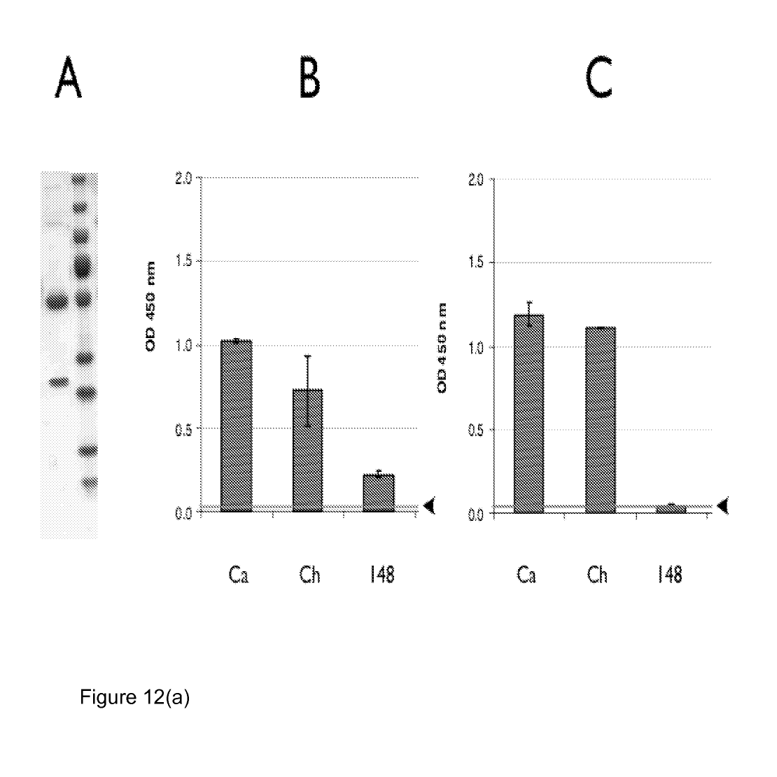

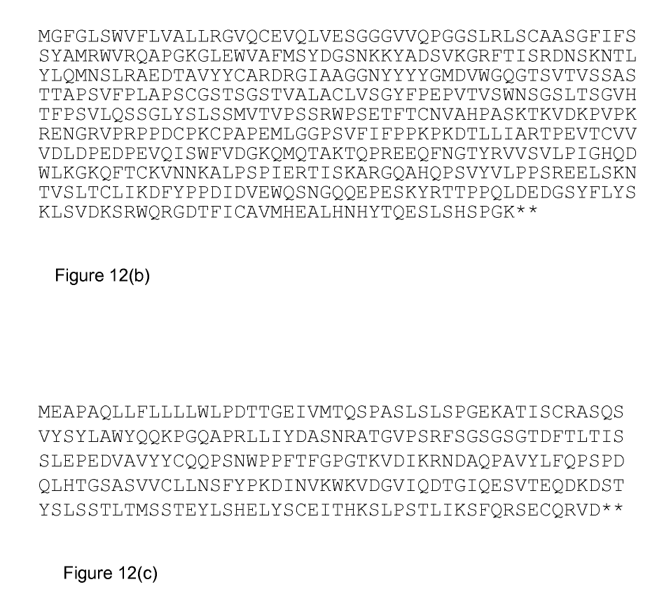

FIG. 12 (a) illustrates a comparison of the caninised and chimeric D2E7 monoclonal antibodies (MAbs) with a further caninised antibody based on anti-human TNF MAb clone 148. The caninised anti-huTNF MAb 148 (SEQ ID NO:18 (ca148-HCB) and SEQ ID NO:19 (ca148-kLC) was expressed in CHO cells and purified using Protein A chromatography (Panel A, left lane). The MAb was tested for binding to human TNF (Panel B) and canine TNF (Panel C) in comparison to caninised (Ca) and chimeric (Ch) D2E7 based MAbs from FIGS. 10 and 11 (background negative control binding is shown by the arrows). It can be seen from panels B and C that the caninised MAb 148 binds to human TNF (Panel B) but not canine TNF (Panel C). Accordingly, the caninised and chimeric MAbs based on D2E7 and the subject of this invention show unexpectedly strong binding to canine TNF equivalent to that to human TNF whereas caninised MAb 148 shows binding to human TNF alone. Therefore, caninised D2E7 based MAbs are surprisingly useful for the treatment of canine diseases mediated by canine TNF. FIGS. 12(b) and 12(c) illustrate the amino acid sequence of a heavy chain of caninised MAb 148 (SEQ ID NO:18) and the amino acid sequence of a light chain of caninised MAb 148 (SEQ ID NO:19) respectively.

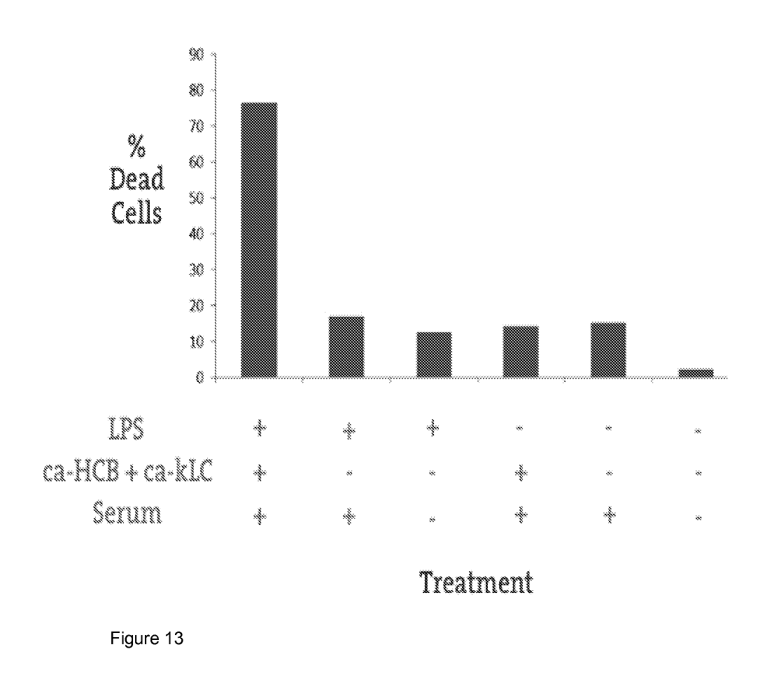

FIG. 13 illustrates the activity of the caninised D2E7 monoclonal antibody in mediating complement killing of TNF expressing cells. RAW cells were treated (columns 1-3) or untreated (columns 4-6) with 10 ng/ml LPS to induce membrane-bound and secreted TNF expression. The cells were subsequently treated with serum in the presence or absence of 10 μg/ml caninised anti-TNF antibody (ca-HCB+ca-kLC). Columns 1 and 4, serum plus antibody; columns 2 and 5, serum only; columns 3 and 6, no serum or antibody. Cell killing was assessed by counting Trypan Blue stained dead cells. Specific cell killing was observed only with the combination of caninised antibody, serum and LPS treatment (column 1). As can be seen in FIG. 13, subsequent treatment with the combination of caninised antibodies plus serum induced specific cell death measured by trypan blue staining, whereas antibody or serum alone could not. Injection of such antibodies to dogs would reduce the number of canine TNF expressing cells in vivo and result in long-term anti-inflammatory effects.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1.-56. (canceled)

57. A method of preparing a caninised antibody or an antigen binding fragment thereof which binds specifically to canine tumour necrosis factor alpha, comprising:
providing a donor antibody from a species other than a canine, wherein the donor antibody has binding specificity for tumour necrosis factor alpha;
comparing each amino acid residue of the amino acid sequence of framework regions of the donor antibody with each amino acid residue present at a corresponding position in the amino acid sequence of framework regions of one or more canine antibodies to identify one or more amino acid residues within the amino acid sequence of the framework regions of the donor antibody that differ from one or more amino acid residues at the corresponding position within the amino acid sequence of framework regions of the one or more canine antibodies; and
substituting the one or more identified amino acid residues in the donor antibody with the one or more amino acid residues present at the corresponding position in the one or more canine antibodies,
wherein the caninised antibody or antigen binding fragment thereof does not contain any amino acid in any position within the framework regions which would be foreign at that position in canines.

58. An antibody or an antigen binding fragment thereof which binds specifically to canine tumour necrosis factor alpha comprising a light chain variable region comprising the amino acid sequence of SEQ ID NO:1 or an amino acid sequence which has an identity of at least 85% thereto and a heavy chain variable region comprising the amino acid sequence of SEQ ID NO:2 or an amino acid sequence which has an identity of at least 85% thereto, wherein the caninised antibody or antigen binding fragment thereof does not contain any amino acid in any position within the framework regions which would be foreign at that position in canines.

59. The antibody or an antigen binding fragment thereof as claimed in claim 58, wherein the antibody comprises a light chain variable region comprising the amino acid sequence of SEQ ID NO:1 and a heavy chain variable region comprising the amino acid sequence of SEQ ID NO:2.

60. The antibody or antigen binding fragment as claimed in claim 58 wherein the antibody comprises a light chain comprising the amino acid sequence of SEQ ID NO:3 or an amino acid sequence which has an identity of at least 85% thereto and a heavy chain comprising the amino acid sequence of SEQ ID NO:5 or an amino acid sequence which has a sequence identity of at least 85% thereto.

61. The antibody or antigen binding fragment as claimed in claim 58 wherein the antibody comprises a light chain comprising the amino acid sequence of SEQ ID NO:3 and a heavy chain comprising the amino acid sequence of SEQ ID NO:5.

62. The antibody or antigen binding fragment as claimed in claim 58 wherein the antibody comprises a light chain comprising the amino acid sequence of SEQ ID NO:8 and a heavy chain comprising the amino acid sequence of SEQ ID NO:9.

63. An antibody or an antigen binding fragment thereof which binds specifically to canine tumour necrosis factor alpha, comprising
a light chain variable region comprising:
an FR1 framework region consisting of or comprising the amino acid sequence of SEQ ID NO:10, or an amino acid sequence which has a sequence identity of at least 85% thereto,
an FR2 framework region consisting of or comprising the amino acid sequence of SEQ ID NO:11, or an amino acid sequence which has a sequence identity of at least 85% thereto,
an FR3 framework region consisting of or comprising the amino acid sequence of SEQ ID NO:12, or an amino acid sequence which has a sequence identity of at least 85% thereto, and
an FR4 framework region consisting of or comprising the amino acid sequence of SEQ ID NO:13, or an amino acid sequence which has a sequence identity of at least 85% thereto,
and a heavy chain variable region comprising:
an FR1 framework region consisting of or comprising the amino acid sequence of SEQ ID NO:14, or an amino acid sequence which has a sequence identity of at least 85% thereto,
an FR2 framework region consisting of or comprising the amino acid sequence of SEQ ID NO:15, or an amino acid sequence which has a sequence identity of at least 85% thereto,
an FR3 framework region consisting of or comprising the amino acid sequence of SEQ ID NO:16, or an amino acid sequence which has a sequence identity of at least 85% thereto, and
an FR4 framework region consisting of or comprising the amino acid sequence of SEQ ID NO:17, or an amino acid sequence which has a sequence identity of at least 85% thereto.

64. The antibody or antigen binding fragment thereof as claimed in claim 63, wherein the light chain variable region comprises:
an FR1 framework region consisting of or comprising the amino acid sequence of SEQ ID NO:10,
an FR2 framework region consisting of or comprising the amino acid sequence of SEQ ID NO:11,
an FR3 framework region consisting of or comprising the amino acid sequence of SEQ ID NO:12, and
an FR4 framework region consisting of or comprising the amino acid sequence of SEQ ID NO:13,
and the heavy chain variable region comprises:
an FR1 framework region consisting of or comprising the amino acid sequence of SEQ ID NO:14,
an FR2 framework region consisting of or comprising the amino acid sequence of SEQ ID NO:15,
an FR3 framework region consisting of or comprising the amino acid sequence of SEQ ID NO:16, and
an FR4 framework region consisting of or comprising the amino acid sequence of SEQ ID NO:17.

65. The antibody or antigen binding fragment as claimed in claim 58 comprising a heavy chain comprising the amino acid sequence selected from the group consisting of SEQ ID NO:4, aglycosylated SEQ ID NO:4, aglycosylated SEQ ID NO:5, aglycosylated SEQ ID NO:6, aglycosylated SEQ ID NO:7 and SEQ ID NO:7, or an amino acid sequence which has a sequence identity of at least 85% thereto.

66. The antibody or antigen binding fragment as claimed in claim 65 wherein the antibody comprises a heavy chain comprising the amino acid sequence selected from the group consisting of SEQ ID NO:5 and SEQ ID NO:6, or an amino acid sequence which has a sequence identity of at least 85% thereto.

67. The antibody or antigen binding fragment as claimed in claim 63 comprising a heavy chain comprising the amino acid sequence selected from the group consisting of SEQ ID NO:4, aglycosylated SEQ ID NO:4, aglycosylated SEQ ID NO:5, aglycosylated SEQ ID NO:6, aglycosylated SEQ ID NO:7 and SEQ ID NO:7, or an amino acid sequence which has a sequence identity of at least 85% thereto.

68. The antibody or antigen binding fragment as claimed in claim 67 wherein the antibody comprises a heavy chain comprising the amino acid sequence selected from the group consisting of SEQ ID NO:5 and SEQ ID NO:6, or an amino acid sequence which has a sequence identity of at least 85% thereto.

69. A pharmaceutical composition comprising a therapeutically effective amount of an antibody or an antigen binding fragment thereof as claimed in claim 58 and at least one pharmaceutically acceptable carrier, excipient or diluent.

70. A pharmaceutical composition comprising a therapeutically effective amount of an antibody or an antigen binding fragment thereof as claimed in claim 63 and at least one pharmaceutically acceptable carrier, excipient or diluent.

71. A method for treating, ameliorating or preventing an immune mediated condition in a canine, comprising administering a therapeutically effective amount of an antibody or antigen binding fragment as claimed in claim 58 to a canine in need thereof.

72. The method as claimed in claim 71 wherein the immune mediated condition is a chronic inflammatory disease or a TNF-alpha related disorder.

73. A method for treating, ameliorating or preventing an immune mediated condition in a canine, comprising administering a therapeutically effective amount of an antibody or antigen binding fragment as claimed in claim 63 to a canine in need thereof.

74. The method as claimed in claim 73 wherein the immune mediated condition is a chronic inflammatory disease or a TNF-alpha related disorder.

75. A kit for the treatment of a canine chronic inflammatory disease comprising an antibody or antigen binding fragment as claimed in claim 58 and instructions for use of the same.

76. A kit for the treatment of a canine chronic inflammatory disease comprising an antibody or antigen binding fragment as claimed in claim 63 and instructions for use of the same.


SEQUENCE LISTING (22 sequences)

SEQ ID NO: 1 - protein, 107 aa, Artificial Sequence
  Caninised anti-TNF Mab VK domain
DIVMTQSPASLSLSQEEKVTITCRASQGIRNYLAWYQQKPGQAPKLLIYAASTLQSGVPSRFSGSGSGTDFTLTISSLEPEDVAVYYCQRYNRAPYTFGQGTKVEIK

SEQ ID NO: 2 - protein, 121 aa, Artificial Sequence
  Caninised anti-TNF MAb VH domain
EVQLVESGGGLVQPGGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSS

SEQ ID NO: 3 - protein, 217 aa, Artificial Sequence
  Caninised anti-TNF MAb kappa light chain
DIVMTQSPASLSLSQEEKVTITCRASQGIRNYLAWYQQKPGQAPKLLIYAASTLQSGVPSRFSGSGSGTDFTLTISSLEPEDVAVYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 4 - protein, 452 aa, Artificial Sequence
  Caninised anti-TNF Mab heavy chain A type constant domain
EVQLVESGGGLVQPGGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 5 - protein, 456 aa, Artificial Sequence
  Caninised anti-TNF Mab heavy chain B type constant domain
EVQLVESGGGLVQPGGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFYPPDIDVEWQSNGQQEPESKYRTTPPQLDEDGSYFLYSKLSVDKSRWQRGDTFICAVMHEALHNHYTQESLSHSPGK

SEQ ID NO: 6 - protein, 454 aa, Artificial Sequence
  Caninised anti-TNF Mab heavy chain C type constant domain
EVQLVESGGGLVQPGGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 7 - protein, 452 aa, Artificial Sequence
  Caninised anti-TNF Mab heavy chain D type constant domain
EVQLVESGGGLVQPGGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFYPPEIDVEWQSNGQPEPESKYHTTAPQLDEDGSYFLYSKLSVDKSRWQQGDTFTCAVMHEALQNHYTDLSLSHSPGK

SEQ ID NO: 8 - protein, 217 aa, Artificial Sequence
  Chimeric anti-TNF MAb. Human VK - canine kappa light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 9 - protein, 402 aa, Artificial Sequence
  Chimeric anti-TNF MAb.  Human VH - canine heavy chain type B constant domain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFYPPDIDVEWQSNGQQEPESKYRT

SEQ ID NO: 10 - protein, 23 aa, Artificial Sequence
  VL FR1
DIVMTQSPASLSLSQEEKVTITC

SEQ ID NO: 11 - protein, 15 aa, Artificial Sequence
  VL FR2
WYQQKPGQAPKLLIY

SEQ ID NO: 12 - protein, 32 aa, Artificial Sequence
  VL FR3
GVPSRFSGSGSGTDFTLTISSLEPEDVAVYYC

SEQ ID NO: 13 - protein, 10 aa, Artificial Sequence
  VL FR4
FGQGTKVEIK

SEQ ID NO: 14 - protein, 27 aa, Artificial Sequence
  VH1 FR1
EVQLVESGGGLVQPGGSLRLSCAASGF

SEQ ID NO: 15 - protein, 14 aa, Artificial Sequence
  VH1 FR2
WVRQAPGKGLEWVS

SEQ ID NO: 16 - protein, 32 aa, Artificial Sequence
  VH1 FR3
RFTISRDNAKNSLYLQMNSLRAEDTAVYYCAK

SEQ ID NO: 17 - protein, 11 aa, Artificial Sequence
  VH1 FR4
WGQGTLVTVSS

SEQ ID NO: 18 - protein, 480 aa, Artificial Sequence
  ca148-HCB
MGFGLSWVFLVALLRGVQCEVQLVESGGGVVQPGGSLRLSCAASGFIFSSYAMRWVRQAPGKGLEWVAFMSYDGSNKKYADSVKGRFTISRDNSKNTLYLQMNSLRAEDTAVYYCARDRGIAAGGNYYYYGMDVWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFYPPDIDVEWQSNGQQEPESKYRTTPPQLDEDGSYFLYSKLSVDKSRWQRGDTFICAVMHEALHNHYTQESLSHSPGK

SEQ ID NO: 19 - protein, 238 aa, Artificial Sequence
  ca148-kLC
MEAPAQLLFLLLLWLPDTTGEIVMTQSPASLSLSPGEKATISCRASQSVYSYLAWYQQKPGQAPRLLIYDASNRATGVPSRFSGSGSGTDFTLTISSLEPEDVAVYYCQQPSNWPPFTFGPGTKVDIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 20 - protein, 452 aa, Artificial Sequence
  Chimeric anti-TNF MAb.  Human VH - canine heavy chain type A constant domain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 21 - protein, 454 aa, Artificial Sequence
  Chimeric anti-TNF MAb.  Human VH - canine heavy chain type C constant domain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 22 - protein, 452 aa, Artificial Sequence
  Chimeric anti-TNF MAb.  Human VH - canine heavy chain type D constant domain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFYPPEIDVEWQSNGQPEPESKYHTTAPQLDEDGSYFLYSKLSVDKSRWQQGDTFTCAVMHEALQNHYTDLSLSHSPGK